(19) United States
(12) Patent Application Publication
(10) Pub. No.: US 2022/0193054 A1
(43) Pub. Date: Jun. 23, 2022

(54) PHARMACEUTICAL COMPOSITION COMPRISING FEXOFENADINE, FAMOTIDINE AND MELATONIN

(71) Applicant: Cellix Bio Private Limited, Hyderabad (IN)

(72) Inventor: Mahesh Kandula, East Godavari District (IN)

(73) Assignee: Cellix Bio Private Limited, Hyderabad (IN)

(21) Appl. No.: 17/488,252

(22) Filed: Sep. 28, 2021

Related U.S. Application Data
(63) Continuation of application No. PCT/IB2021/057998, filed on Sep. 2, 2021.

(30) Foreign Application Priority Data
Dec. 19, 2020 (IN) 202041055350
Jan. 23, 2021 (IN) 202141003284
Feb. 17, 2021 (IN) 202141006680
Apr. 8, 2021 (IN) 202141016620

(51) Int. Cl. A61K 31/445; A61K 31/426; A61K 31/4045; A61K 9/20

(52) U.S. Cl. CPC A61K 31/445; A61K 9/2013; A61K 31/4045; A61K 31/426

(57) ABSTRACT

The present disclosure provides a pharmaceutical composition comprising Fexofenadine or salt or hydrates or solvates thereof, Famotidine or salt or hydrates or solvates thereof, and Melatonin or salt or hydrates or solvates thereof. The compositions of the present disclosure may find utility in treatment of urticaria, atopic dermatitis, pruritus and the like acute or chronic allergic reactions and/or dermatological diseases/conditions, and sleep disorders. Aspects of the present disclosure also relates to a method of treating inflammation associated with COVID 19, gastrointestinal diseases, urticaria, atopic dermatitis, pruritus and the like acute or chronic allergic reactions and/or dermatological diseases/conditions, and sleep disorders using the advantageous compositions of the present disclosure.

### TECHNICAL FIELD

The present disclosure generally relates to the field of pharmaceutical compositions. In particular, the present disclosure provides a pharmaceutical composition comprising Fexofenadine or salt or hydrates or solvates thereof, Famotidine or salt or hydrates or solvates thereof, and Melatonin or salt or hydrates or solvates thereof. The compositions of the present disclosure may find utility in treatment of urticaria, atopic dermatitis, pruritus and the like acute or chronic allergic reactions and/or dermatological diseases/conditions, and sleep disorders.

### BACKGROUND

Allergic reactions or hypersensitivity is defined as an exaggerated or inappropriate state of normal immune response with onset of adverse effects on the body. These are a form of antigen-antibody reaction that occurs when immune system of a subject responds abnormally to common substances such as pollen, dust, certain foods and drugs. This results in high secretion of histamine from immune cells causing severe anaphylactic symptoms. Inflammation during the allergies occurs due to a complex interaction between several inflammatory cells, including mast cells, basophils, lymphocytes, dendritic cells, eosinophil, and sometimes neutrophils. These cells produce multiple inflammatory mediators, including lipids, purines, cytokines, chemokine, and reactive oxygen species.

Urticaria, commonly referred to as hives, is a kind of skin rash notable for pale red, raised, itchy bumps. Burning or stinging sensation may also be present. Hives are frequently caused by allergic reactions; however, there are many non-allergic causes. Most cases of hives lasting less than six weeks (acute urticaria) are the result of an allergic trigger. Chronic urticaria (hives lasting longer than six weeks) is rarely due to an allergy. The majority of chronic hives cases have an unknown (idiopathic) cause. In as many as 30-40% of patients with chronic idiopathic urticaria, it is caused by an autoimmune reaction.

Atopic dermatitis is a chronic disease that affects the skin. In atopic dermatitis, the skin becomes extremely itchy and inflamed, causing redness, swelling, cracking, weeping, crusting, and scaling. It is the most common of the many types of eczema.

Pruritus, or itch, is a sensation that stimulates the desire or reflex to scratch, which can be either generalized or localized. The cause of pruritus is not fully understood. Proposed contributors to the pathogenesis of pruritus may include anemia or other manifestation of erythropoietin deficiency, histamine release from skin mast cells, skin dryness, secondary hyperparathyroidism, hyperphosphatemia with increased calcium phosphate deposition in the skin and alterations in the endogenous opioidergic system with overexpression of opioid receptors.

To solve the above existing problems, significant efforts have been put forth by the researchers to find products for treating such disorders. However, none of the existing approaches seem to satisfy the existing needs. A need is also felt of improved formulations that are easy to administer and aids in improving patient compliance. The present disclosure satisfies the existing needs, at least in part, and overcomes one or more disadvantages of the conventional approaches.

### OBJECTS

One of the objects of the present disclosure is to provide a pharmaceutical composition that may overcome the limitations associated with the conventional compositions.

Another object of the present disclosure is to provide a composition that exhibits superior storage stability and functional reciprocity.

Further object of the present disclosure is to provide a composition that is easy to prepare and is economical.

Yet another object of the present disclosure is to provide a pharmaceutical composition to deliver as an immediate release or modify or control the delivery rate of different active agents in the formulation.

Still another object of the present disclosure is to deliver the active agents either simultaneously or concurrently or concomitantly to a subject for the treatment of a disease.

### SUMMARY

The present disclosure generally relates to the field of pharmaceutical compositions. In particular, the present disclosure provides a pharmaceutical composition comprising Fexofenadine or salt or hydrates or solvates thereof, Famotidine or salt or hydrates or solvates thereof, and Melatonin or salt or hydrates or solvates thereof.

In an embodiment, the composition comprises Fexofenadine or salt or hydrates or solvates thereof, Famotidine or salt or hydrates or solvates thereof and Melatonin or salt or hydrates or solvates thereof in a weight ratio ranging from 1:1:1 to 100:50:1. In an embodiment, the composition is a fixed dose combination.

In an embodiment, the composition comprises Fexofenadine or salt or hydrates or solvates thereof, Famotidine or salt or hydrates or solvates thereof and Melatonin or salt or hydrates or solvates thereof in a weight ratio ranging from 4:2:1 to 50:25:1.

In an embodiment, Fexofenadine or salt or hydrates or solvates thereof is present in an amount ranging from 20 mg to 500 mg. In embodiment, Famotidine or salt or hydrates or solvates thereof is present in an amount ranging from 10 mg to 100 mg. In an embodiment, Melatonin or salt or hydrates or solvates thereof is present in an amount ranging from 1 mg to 80 mg.

In an embodiment, the composition includes: Fexofenadine or salt or hydrates or solvates thereof in an amount ranging from 20 mg to 500 mg, Famotidine or salt or hydrates or solvates thereof in an amount ranging from 10 mg to 100 mg, and Melatonin or salt or hydrates or solvates thereof in an amount ranging from 1 mg to 80 mg. In one embodiment, Fexofenadine is present as Fexofenadine hydrochloride.

The composition also includes a pharmaceutically acceptable excipient. The pharmaceutically acceptable excipient is selected from any or a combination of: a diluent, an anti-oxidant, a preservative, an alkalizing agent, a buffering agent, a disintegrant, a binder, an anti-foaming agent, a solvent, a glidant, a lubricant, a flavoring agent, a coating agent, a rate controlling polymer or non-polymer, a zinc salt, a fatty acid or derivative thereof, an amino acid or metabolites or amino acid derivatives, a bulking agent, an anti-tacking agent, an emulsifier, a surfactant, a plasticizer and a stabilizer.

In yet another embodiment, there is disclosed a fixed dose pharmaceutical composition comprising: Fexofenadine or salt or hydrates or solvates thereof, Famotidine or salt or hydrates or solvates thereof and Melatonin or salt or hydrates or solvates thereof. In an embodiment, the composition comprises an intra-granular portion and an extra-granular portion, wherein the intra-granular portion comprises Fexofenadine or salt or hydrates or solvates thereof, Famotidine or salt or hydrates or solvates thereof, Melatonin or salt or hydrates or solvates thereof and a pharmaceutically acceptable excipient, and the extra-granular portion comprises a pharmaceutically acceptable excipient.

In yet another embodiment, there is disclosed a fixed dose pharmaceutical composition comprising: Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, wherein the intra-granular portion comprises Famotidine or salt or hydrates or solvates thereof, Melatonin or salt or hydrates or solvates thereof and a pharmaceutically acceptable excipient, and the extra-granular portion comprises Fexofenadine or salt or hydrates or solvates thereof and a pharmaceutically acceptable excipient.

In yet another embodiment, there is disclosed a fixed dose pharmaceutical composition comprising: Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, wherein the intra-granular portion comprises Fexofenadine or salt or hydrates or solvates thereof, Melatonin or salt or hydrates or solvates thereof and a pharmaceutically acceptable excipient, and the extra-granular portion comprises Famotidine or salt or hydrates or solvates thereof and a pharmaceutically acceptable excipients.

In yet another embodiment, there is disclosed a fixed dose pharmaceutical composition comprising: Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, wherein the intra-granular portion comprises Fexofenadine or salt or hydrates or solvates thereof, Famotidine or salt or hydrates or solvates thereof and a pharmaceutically acceptable excipient, and the extra-granular portion comprises Melatonin or salt or hydrates or solvates thereof and a pharmaceutically acceptable excipient.

In yet another embodiment, the composition comprises an intra-granular portion and an extra-granular portion, wherein the intra-granular portion comprises Fexofenadine HCl in an amount of 60 mg, Famotidine in an amount of 40 mg, Melatonin in an amount of 4 mg, and a pharmaceutically acceptable excipient, and the extra-granular portion comprises a pharmaceutically acceptable excipient.

In yet another embodiment, the composition comprises an intra-granular portion and an extra-granular portion, wherein the intra-granular portion comprises Fexofenadine in an amount of 60 mg, Famotidine in an amount of 40 mg, Melatonin in an amount of 4 mg, and a pharmaceutically acceptable excipient, and the extra-granular portion comprises a pharmaceutically acceptable excipient.

In yet another embodiment, the composition comprises an intra-granular portion and an extra-granular portion, wherein the intra-granular portion comprises Fexofenadine HCl in an amount of 120 mg, Famotidine in an amount of 40 mg, Melatonin in an amount of 3 mg, and a pharmaceutically acceptable excipient, and the extra-granular portion comprises a pharmaceutically acceptable excipient.

In yet another embodiment, the composition comprises an intra-granular portion and an extra-granular portion, wherein the intra-granular portion comprises Fexofenadine in an amount of 120 mg, Famotidine in an amount of 40 mg, Melatonin in an amount of 3 mg, and a pharmaceutically acceptable excipient, and the extra-granular portion comprises a pharmaceutically acceptable excipient.

In an embodiment, the portions are compressed together to obtain a tablet dosage form, optionally coated with a seal coat. In an embodiment, the seal coat is an aqueous seal coat.

### DETAILED DESCRIPTION

The present disclosure generally relates to the field of pharmaceutical compositions.

As used herein, the following terms and phrases shall have the meanings set forth below. Unless defined otherwise, all technical and scientific terms used herein have the same meaning as commonly understood to one of ordinary skill in the art. It is also understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, “an active agent” or “an active ingredient” refers not only to a single active agent but also to a combination of two or more different active agents, “a dosage form” refers to a combination of dosage forms as well as to a single dosage form, and the like.

The term “active agent” or “therapeutic agent”, encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds.

The term “combination therapy” or “combined treatment” or “in combination” as used herein denotes any form of simultaneous or concurrent or concomitantly or co-administration of active agents for treating urticaria, atopic dermatitis, pruritus and the like acute or chronic allergic reactions and/or dermatological diseases/conditions and sleep disorders.

The terms “treating” and “treatment” as used herein refers to reduction in severity and/or frequency of symptoms, elimination of symptoms and/or underlying cause, and improvement or remediation of damage caused thereby. Thus, “treating” a subject/patient as described herein encompasses treating urticaria, atopic dermatitis, pruritus, Covid-19 associated inflammatory complications such as Cytokines release etc., as well as gastrointestinal diseases such as GERD, gastritis, severe GERD and the like acute or chronic allergic reactions and/or dermatological diseases/conditions, gastrointestinal and sleep disorders.

The term “dosage form” denotes any form of a pharmaceutical composition that contains an amount of active agent sufficient to elicit a desired therapeutic response.

The term “controlled release” refers to a drug-containing formulation or fraction thereof in which release of the drug is not immediate. The term “controlled release” as used herein includes sustained release, non-immediate release and delayed release formulations.

The term “immediate release” is used herein in its conventional sense to refer to a drug formulation that provides for immediate release of the drug(s) contained therein.

The term “sustained release” (synonymous with “extended release”) is used in its conventional sense to refer to a drug formulation that provides for gradual release of a drug over an extended period of time.

The term “pharmaceutically acceptable” means the material incorporated into a pharmaceutical composition that can be administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

“Pharmacologically active” (or simply “active”) as in a pharmacologically active derivative or analog, refers to a derivative or analog having the same type of pharmacological activity as the parent compound and approximately equivalent in degree.

The present disclosure provides a pharmaceutical composition comprising: Fexofenadine or salt or hydrates or solvates thereof, Famotidine or salt or hydrates or solvates thereof, and Melatonin or salt or hydrates or solvates thereof. In an embodiment, the composition is a fixed dose combination. The compositions of the present disclosure may find utility in treatment of urticaria, atopic dermatitis, pruritus and the like acute or chronic allergic reactions and/or dermatological diseases/conditions and sleep disorders.

In an embodiment, the composition comprises Fexofenadine or salt or hydrates or solvates thereof, Famotidine or salt or hydrates or solvates thereof and Melatonin or salt or hydrates or solvates thereof in a weight ratio ranging from 1:1:1 to 100:50:1.

In an embodiment, the composition comprises Fexofenadine or salt or hydrates or solvates thereof, Famotidine or salt or hydrates or solvates thereof and Melatonin or salt or hydrates or solvates thereof in a weight ratio ranging from 1:1:1 to 50:25:1.

In an embodiment, the composition comprises Fexofenadine or salt or hydrates or solvates thereof, Famotidine or salt or hydrates or solvates thereof and Melatonin or salt or hydrates or solvates thereof in a weight ratio ranging from 4:2:1 to 50:25:1.

In an embodiment, the composition comprises Fexofenadine or salt or hydrates or solvates thereof, Famotidine or salt or hydrates or solvates thereof and Melatonin or salt or hydrates or solvates thereof in a weight ratio ranging from 10:7:1 to 50:15:1.

In an embodiment, the composition comprises Fexofenadine or salt or hydrates or solvates thereof, Famotidine or salt or hydrates or solvates thereof, and Melatonin or salt or hydrates or solvates thereof in a weight ratio of 15:10:1.

In an embodiment, the composition comprises Fexofenadine or salt or hydrates or solvates thereof, Famotidine or salt or hydrates or solvates thereof, and Melatonin or salt or hydrates or solvates thereof in a weight ratio of 120:40:3.

In an embodiment, Fexofenadine or salt or hydrates or solvates thereof is present in the composition in an amount ranging from 20 mg to 500 mg. In embodiment, Famotidine or salt or hydrates or solvates thereof is present in an amount ranging from 10 mg to 100 mg. In an embodiment, Melatonin or salt or hydrates or solvates thereof is present in an amount ranging from 1 mg to 80 mg.

In an embodiment, Fexofenadine or salt or hydrates or solvates thereof is present in the composition in an amount of 20 mg to 500 mg. Alternatively, Fexofenadine or salt or hydrates or solvates thereof is present in an amount of 20 mg to 200 mg. Alternatively, Fexofenadine or salt or hydrates or solvates thereof is present in an amount of 30 mg to 150 mg. In an embodiment, the amount of Fexofenadine or salt or hydrates or solvates thereof in the composition is 60 mg. In an embodiment, the amount of Fexofenadine or salt or hydrates or solvates thereof in the composition is 120 mg. In an embodiment, Fexofenadine is present as Fexofenadine hydrochloride.

In an embodiment, Famotidine or salt or hydrates or solvates thereof is present in the composition in an amount of 10 mg to 100 mg. Alternatively, Famotidine or salt or hydrates or solvates thereof is present in an amount of 20 mg to 80 mg. Still alternatively, Famotidine or salt or hydrates or solvates thereof is present in an amount of 30 mg to 50 mg. In an embodiment, the amount of Famotidine or salt or hydrates or solvates thereof in the composition is 40 mg.

The composition includes Melatonin or salt or hydrates or solvates thereof in an amount ranging from 1 mg to 80 mg. Alternatively, Melatonin or salt or hydrates or solvates thereof is present in an amount of 2 mg to 50 mg. Alternatively, Melatonin or salt or hydrates or solvates thereof is present in an amount of 2 mg to 40 mg. Still alternatively, Melatonin or salt or hydrates or solvates thereof is present in an amount of 2 mg to 10 mg. In an embodiment, the amount of Melatonin or salt or hydrates or solvates thereof in the composition is 3 mg. In an embodiment, the amount of Melatonin or salt or hydrates or solvates thereof in the composition is 4 mg.

In an embodiment, any of the three active agents fexofenadine, famotidine and melatonin are given simultaneously as individual formulations/compositions or two active agents are given in a combination and one active agent is given simultaneously along with the two active agents in combination to a subject.

In another embodiment, fexofenadine, famotidine and melatonin are given simultaneously as individual formulations/compositions. Alternatively, famotidine and fexofenadine are given in a combination as a formulation and melatonin is given simultaneously as in a separate formulation. Still alternatively, fexofenadine and melatonin are given in a combination as a formulation and famotidine is given simultaneously as a separate formulation. Still alternatively, famotidine and melatonin are given in a combination as a formulation and fexofenadine is given simultaneously as a separate formulation.

In another embodiment, active agents fexofenadine, famotidine and melatonin are formulated as individual formulations/compositions in titrated strengths or binary mixture compositions or a fixed dosage combination of either 2 active agents and 1 active is given simultaneously for the treatment of a disease in a subject.

In an embodiment, any of the three active agents famotidine, melatonin and fexofenadine are given concomitantly as individual formulations/compositions or two active agents are given in a combination and one active agent is given concomitantly as a separate formulation to a subject.

In another embodiment, famotidine, fexofenadine and melatonin are given concomitantly as individual formulations/compositions or famotidine and fexofenadine are given in a combination as a formulation and melatonin is given concomitantly as a separate formulation or fexofenadine and melatonin are given in a combination as a formulation and famotidine is given concomitantly as a separate formulation or famotidine and melatonin are given in a combination as a formulation and fexofenadine is given concomitantly as a separate formulation to a subject.

In another embodiment, active agents famotidine, fexofenadine and melatonin are formulated as individual formulations/compositions in titrated strengths or binary mixture compositions or a fixed dosage combination of 2 active agents and the remainder of 1 active agent is given concomitantly for the effective treatment of a disease in a subject.

The composition also includes a pharmaceutically acceptable excipient. The pharmaceutically acceptable excipient is selected from any or a combination of: a diluent, an anti-oxidant, a preservative, an alkalizing agent, a buffering agent, a disintegrant, a binder, an anti-foaming agent, a solvent, a glidant, a lubricant, a flavoring agent, a sweetener, a coating agent, a rate controlling polymer or non-polymer, a zinc salt, a fatty acid or derivative thereof, an amino acid or metabolites or amino acid derivatives, a bulking agent, an anti-tacking agent, an emulsifier, a surfactant, a plasticizer and a stabilizer.

In an embodiment, the diluent(s) include(s), but not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, and magnesium aluminum silicate and mixtures thereof.

In an embodiment, the anti-oxidant(s) and preservative(s) include(s), but not limited to, L-Carnosine, vitamin A, vitamin E, vitamin C, retinyl palmitate, and selenium, citric acid, sodium citrate, methyl paraben, propyl paraben, p-hydroxybenzoic acid esters, sorbic acid, benzoic acid, propionic acid or salts thereof; Alcohols such as benzyl alcohol, butanol or ethanol, isopropyl alcohol, and quaternary ammonium compounds such as benzalkonium chloride, sodium benzoate and mixtures thereof.

In an embodiment, the alkalizing agent(s) include(s), but not limited to, ammonia solution NF, Ammonium Carbonate NF, Diethanolamine NF, monoethanolamine, Potassium Hydroxide NF, Sodium Bicarbonate USP, Sodium Borate NF, Sodium Carbonate NF, Sodium Hydroxide NF, sodium Phosphate Dibasic USP, trolamine NF, calcium carbonate, magnesium carbonate, magnesium oxide, magnesium hydroxide, magnesium trisilicate, aluminum hydroxide, aluminum carbonate, magnesium aluminium silicate hydrate, potassium bicarbonate, sodium bicarbonate, sodium citrate, potassium citrate, aluminum sulfate, calcium carbonate and mixtures thereof.

In an embodiment, the buffering agent(s) include(s), but not limited to, a bicarbonate salt of alkali earth metal, amino acids, an acid salt of an amino acid, an alkali salt of an amino acid and mixture thereof.

In an embodiment, the disintegrant(s) include(s), but not limited to Croscarmellose sodium, microcrystalline cellulose, low-substituted hydroxypropyl cellulose, alginic acid and alginates, modified starches, sodium starch glycolate, sodium carboxy methyl cellulose, carboxymethyl cellulose calcium, polyvinylpyrrolidone, docusate sodium, guar gum and mixtures thereof.

In an embodiment the binder(s) include(s), but not limited to, hypromellose (or hypromellose 5 cps), polyvinyl pyrrolidone, copolymers of vinyl pyrrolidone with other vinylderivatives, hydroxypropyl cellulosic derivatives (such as methylcellulose, carboxymethyl cellulose, hydroxyethyl cellulose, hydroxyethylmethyl cellulose, hydroxypropylcellulose etc), polyacrylates (such as Carbopol, polycarbophil, etc), Povidone (all grades), Polyox of any molecular weight or grade, irradiated or not, maize starch, povidone, copovidone, corn starch, starch, polyvinylpyrrolidone (PVP), microcrystalline cellulose, powdered acacia, gelatin, guar gum, carbomer such as carbopol, polymethacrylates, starch, heavy magnesium oxide and mixtures thereof.

In an embodiment the anti-foaming agent(s) include(s), but not limited to, alcohols such as cetostearyl alcohol, insoluble oils such as castor oil, stearates, polydimethylsiloxanes and other silicones derivatives, ethers, paraffin oil, paraffin wax, glycols, simethicone (or simethicone 30% emulsion) and mixtures thereof.

In an embodiment, solvent(s) include(s), but not limited to, methanol, ethanol, n-propanol, isopropanol, hexane, heptane, petroleum ether, cyclohexane, diethyl ether, diisopropyl ether, ethyl acetate, methyl acetate, ethyl formate, methyl formate, isobutyl acetate, n-butyl acetate, methylene chloride, ethylene chloride, chloroform, carbon tetrachloride, acetone, ethyl methyl ketone, diisobutyl ketone, methyl isobutyl ketone, 1,4-dioxane, toluene, ammonia solution, glacial acetic acid, ammonium hydroxide, sodium hydroxide, calcium hydroxide, calcium carbonate, potassium hydroxide, potassium carbonate, water and mixtures thereof.

In an embodiment, the glidant(s) include(s), but are not limited to, colloidal silicon dioxide, stearic acid, talk, aluminium silicate and mixtures thereof.

In an embodiment, the lubricant(s) include(s), but not limited to, stearic acid, magnesium stearate, sodium stearyl fumarate, sodium lauryl sulphate, magnesium lauryl sulphate, fumaric acid, glyceryl palmitostearate, zinc stearate, calcium stearate, silica, talc, polyethylene glycol, paraffin and mixtures thereof.

In an embodiment, the flavoring agent(s) include(s), but not limited, cherry, maple, pineapple, orange, raspberry, banana-vanilla, peppermint, butterscotch, strawberry, vanilla, apricot, cinnamon, honey, lime, peach-orange, peach-rum, raspberry, wild cherry, mint and mixtures thereof.

In an embodiment, coating agent(s) include(s), but not limited to, Cellulosics, such as hydroxypropyl methyl cellulose (HPMC), methy ethylcellulose (MEC), carboxymethyl celluolose (CMC), carboxymethyl ethylcelluolose (CMEC), hydroxyethyl cellulose (HEC), Hydroxypropyl cellulose (HPC), cellulose acetate phthalate (CAP), hydroxypropyl methyl cellulose phthalate (HPMCP), hydroxypropyl methyl cellulose acetate succinate (HPMCAS), hypromellose, povidone, copovidone, and ethyl cellulose (EC); Vinyls, such as polyvinyl alcohol; Acrylics, such as methacrylic acid/ethylacrylate copolymers; Natural derivatives, such as shellac or alginate and mixtures thereof.

In an embodiment, the rate controlling polymer(s) include(s), but not limited to, cellulose acetate, alkyl celluloses, hydroxyalkyl, acrylic polymers, copolymers dialkylphthalates, dibutyl phthalate, microcrystalline wax and mixtures thereof.

In an embodiment, the rate controlling non-polymer(s) include(s), but not limited to, fat, wax, fatty acid, fatty acid ester, long chain monohydric alcohol or their ester and mixtures thereof.

In an embodiment, zinc salt(s) include(s), but not limited to, zinc oxide, zinc stearate, zinc L-carnosine, zinc acetate, zinc chloride, zinc bromide, zinc fluoride, zinc hexafluorosilicate, zinc iodide, zinc molybdate, zinc nitrate, zinc molybdite, zinc oxalate, zinc perchlorate, zinc tetrafluoroborate, zinc sulfate and mixtures thereof.

In an embodiment, the fatty acid(s) or derivatives thereof include(s), but not limited to, fatty acids with C1 to C30 carbons, which includes long chain fatty acids; saturated or unsaturated fatty acids and derivatives thereof (monounsaturated fatty acids (MUFAs) C18:1n-12c, C16:1n-5, C16:4n-1 and the polyunsaturated fatty acids (PUFAs) C16:3n-4, C20:3n-3, C20:4n-6, C21:5n-3 and C18:2n-9c, 12t); hydrogenated fatty acids; fatty acid glycerides; polyoxyethylated oleic glycerides; monoglycerides and diglycerides; mono-, bi- or tri-substituted glycerides; glycerol mono-oleate esters; glycerol mono-caprate; glyceryl monocaprylate; dicaprylate; laurate, monolaurate; glyceryl palmitostearate; glyceryl behenate; diethyleneglycol palmitostearate; polyethyleneglycol stearate; polyoxyethyleneglycol palmitostearate; glyceryl mono palmitostearate; cetyl palmitate; polyethyleneglycol palmitostearate; dimethylpolysiloxane; mono- or di-glyceryl behenate; fatty acid derivatives such as diglyceryl lauryl fumarate (DGLF), diglyceryl lauryl succinate, diglyceryl capryl succinate, diglyceryl capryl fumarate; fatty alcohols associated with polyethoxylate fatty alcohols; cetyl alcohol; octyldodecanol; myristyl alcohol; isopropyl myristate, isopropyl palmitate, stearic acid, lauric acid, EPA, DHA, linoleic acid, linolenic acid, stearyl alcohol and mixture thereof. In an embodiment, diglyceryl lauryl fumarate (DGLF), diglyceryl lauryl succinate, diglyceryl capryl succinate, or diglyceryl capryl fumarate are used in the composition to delay disintegration and/or absorption and thereby provide sustained action over a longer period.

In an embodiment, the amino acids or metabolites or amino acid derivatives include(s), but not limited to, glycine, glutamine, asparagine, arginine, lysine in biologically active enantiomeric forms, L-carnosine, L-carnitine, choline, betaine, taurine, glycosaminoglycans including hyaluronic acid, chondroitin sulfate, glucosamine, L-glucosamine, heparins and mixtures thereof.

In an embodiment, the bulking agent(s) include(s), but not limited to, lactose USP, Starch 1500, mannitol, erythritol, sorbitol, maltodextrin, malitol or other non-reducing sugars; microcrystalline cellulose (e.g., Avicel), dibasic calcium phosphate (anhydrous or dihydrate), sucrose, etc. and mixtures thereof.

In an embodiment, the anti-tacking agent(s) include(s), but not limited to, stearates; stearic acid; vegetable oil; waxes; a blend of magnesium stearate and sodium lauryl sulfate; sodium benzoate; sodium acetate and mixtures thereof.

In an embodiment, the surfactant(s) and emulsifier(s) include(s), but not limited to, ionic or non-ionic surfactants and emulsifiers, poloxamers, polyethylene glycols, polyethylene glycol monostearate, polysorbates, sodium lauryl sulfate, polyethoxylated, hydrogenated castor oil and mixtures thereof.

In an embodiment, the plasticizer(s) include(s), but are not limited to, diethyl phthalate, triethyl citrate, acetyl tributyl citrate, dibutyl phthalate, triacetin, propylene glycol, polyethylene glycol, dichloromethane, acetone, ethanol, methanol, isopropyl alcohol, water and mixtures thereof.

In an embodiment, the stabilizer(s) include(s), but not limited to, gums, agar, taste masking agents like acrylic polymers, copolymers of acrylates, celluloses, resins and mixtures thereof.

In an embodiment, the sweetener(s) include(s), but not limited to, mannitol, sorbitol, polyethylene glycol (PEG) 6000 and 8000, Emdex, Nu-tab, Sweetrex, Mola-tab, Hony-tab, Sugartab, non-sugar sweetening agents such as aspartame, sorbitol, xylitol, isomalt, saccharin, sodium saccharin, calcium saccharin, sucralose, acesulfame-K, steviol, steviosin, mannitol, erythritol, lactitol, and sugar sweetening agents such as sucrose, fructose, dextrose and mixtures thereof.

Although several embodiments of the present disclosure names few of the commonly used excipients, any other excipient known to or appreciated by a skilled person can also be used to realize the advantageous compositions of the present disclosure. Examples of useful excipients which can optionally be added to the composition are described in the Handbook of Pharmaceutical Excipients, 3rd edition, Edited by A. H. Kibbe, Published by: American Pharmaceutical Association, Washington D.C., ISBN: 0-917330-96-X, and in Handbook of Pharmaceutical Excipients (4th edition), Edited by Raymond C Rowe—Publisher: Science and Practice.

Depending on the intended mode of administration, the pharmaceutical composition may be formatted as a solid, semi-solid or liquid dosage form. Non-limiting examples of dosage forms includes tablet, lozenge, capsule, caplet, modified release tablet or lozenge, suspension, solution, emulsion, suppository, granules, pellets, beads, powder, aerosol sprays (oral, nasal, dermal), cream, ointment, lotion, patches, pre-filled syringe, pre-filled pen, gel, tablet in tablet, bilayer tablet, trilayer tablet, inlay tablet, capsule in capsule, tablet(s) in capsule, granules and/or pellets in-capsule, pellets and tablet in capsules and the likes.

In an embodiment, there is disclosed a fixed dose pharmaceutical composition comprising Fexofenadine or salt or hydrates or solvates thereof, Famotidine or salt or hydrates or solvates thereof, and Melatonin or salt or hydrates or solvates thereof, wherein the composition is formulated into a tablet dosage form. The tablet may be a monolayer tablet comprising Famotidine or salt or hydrates or solvates thereof, Fexofenadine or salt or hydrates or solvates thereof and Melatonin or salt or hydrates or solvates thereof in a single uniform layer. The tablet may be a bilayer tablet comprising Fexofenadine or salt or hydrates or solvates thereof, Famotidine or salt or hydrates or solvates thereof and Melatonin or salt or hydrates or solvates thereof in any of a first layer and a second layer. Alternatively, the tablet may be a trilayer tablet comprising Fexofenadine or salt or hydrates or solvates thereof, Famotidine or salt or hydrates or solvates thereof and Melatonin or salt or hydrates or solvates thereof in any of a first layer, a second layer and a third layer. Alternatively, the tablet may be a press-coated tablet, i.e. a small tablet and a granulation or a blend is compressed together to one large press-coated tablet. All types of the tablets mentioned hereinbefore may be without a coating or may have one or more coatings, in particular film-coatings.

The tablet-in-tablet dosage form may be prepared by compressing active ingredients with one or more rate controlling polymer or non-polymer to form a core extended release tablet; and compressing active ingredients optionally along with one or more pharmaceutically acceptable excipient onto said core tablet to form compressed tablet that causes immediate release of the active ingredients.

In another embodiment of the present invention, the pharmaceutical composition is formulated as an inlay tablet. Inlay tablets are tablets, wherein inner tablet is positioned within a comparatively larger outer tablet in such a way that at least one surface of the inner tablet is not in contact with outer tablet. Inlay tablet dosage form includes: (a) an inner inlayed tablet comprising active ingredients and excipient(s) that causes extended release; and (b) an outer tablet comprising active ingredients along with excipient(s) to cause immediate release.

In another embodiment, the present disclosure embraces capsule-in-capsule formulations, wherein smaller size capsule is encapsulated into a larger capsule. Capsule-in-capsule consists of an external capsule and internal capsule (inner capsule) located therein. It is preferred that smaller size capsule is filled with active ingredients and excipients so as to cause extended release while larger capsule is filled, optionally, with active ingredients along with excipients for immediate release.

The tablet of the present disclosure may be monolithic that means having a homogenous matrix of active ingredient and pharmaceutically acceptable excipients. Alternatively, the tablet may be formed as a bilayer, wherein the one layer is having active ingredients along with pharmaceutically acceptable excipients and other layer is having pharmaceutically acceptable excipients. Alternatively, both layer of bilayer tablet may contain active ingredients.

The composition can be made by different manufacturing processes such as by direct compression, wet granulation, dry granulation, melt granulation, melt congealing, extrusion and the likes. The composition cores may be mono or multi-layer(s) and can be coated with appropriate overcoats as known in the art. Wet granulation involves formation of granules using active ingredient and one or more pharmaceutically acceptable excipients and this portion can be termed as intra-granular portion. These granules are then lubricated with blend of excipients comprising lubricant and this lubricant blend is then compressed to form a tablet. The portion outside the granules can be referred as extra-granular portion. Direct compression on the other hand requires only that the active ingredient is blended with one or more pharmaceutically acceptable excipients before compression and then compressed into tablet. The preferred way for making the composition of the present disclosure is wet granulation.

In an embodiment, there is disclosed a fixed dose pharmaceutical composition comprising Fexofenadine or salt or hydrates or solvates thereof, Famotidine or salt or hydrates or solvates thereof, and Melatonin or salt or hydrates or solvates thereof, wherein the composition comprises an intra-granular portion and an extra-granular portion, wherein the intra-granular portion comprises Fexofenadine or salt or hydrates or solvates thereof, Famotidineor salt or hydrates or solvates thereof, Melatonin or salt or hydrates or solvates thereof and a pharmaceutically acceptable excipient, and the extra-granular portion comprises a pharmaceutically acceptable excipient.

In another embodiment, there is disclosed a fixed dose pharmaceutical composition comprising Fexofenadine or salt or hydrates or solvates thereof, Famotidine or salt or hydrates or solvates thereof, and Melatonin or salt or hydrates or solvates thereof, wherein the composition comprises an intra-granular portion and an extra-granular portion, and wherein the intra-granular portion comprises Famotidine or salt or hydrates or solvates thereof, Melatonin or salt or hydrates or solvates thereof and a pharmaceutically acceptable excipient, and the extra-granular portion comprises Fexofenadine or salt or hydrates or solvates thereof and a pharmaceutically acceptable excipient.

In yet another embodiment, there is disclosed a fixed dose pharmaceutical composition comprising Fexofenadine or salt or hydrates or solvates thereof, Famotidine or salt or hydrates or solvates thereof, and Melatonin or salt or hydrates or solvates thereof, wherein the composition comprises an intra-granular portion and an extra-granular portion, and wherein the intra-granular portion comprises Fexofenadine or salt or hydrates or solvates thereof, Melatonin or salt or hydrates or solvates thereof and a pharmaceutically acceptable excipient, and the extra-granular portion comprises Famotidine or salt or hydrates or solvates thereof and a pharmaceutically acceptable excipients.

In yet another embodiment, there is disclosed a fixed dose pharmaceutical composition comprising Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, and wherein the intra-granular portion comprises Fexofenadine or salt or hydrates or solvates thereof and Famotidine or salt or hydrates or solvates thereof and a pharmaceutically acceptable excipient, and the extra-granular portion comprises Melatonin or salt or hydrates or solvates thereof and a pharmaceutically acceptable excipient.

In yet another embodiment, there is disclosed a fixed dose pharmaceutical composition comprising Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, and wherein the intra-granular portion comprises Fexofenadine or salt or hydrates or solvates thereof and a pharmaceutically acceptable excipient, and the extra-granular portion comprises Melatonin or salt or hydrates or solvates thereof, Famotidine or salt or hydrates or solvates thereof and a pharmaceutically acceptable excipient.

In yet another embodiment, there is disclosed a fixed dose pharmaceutical composition comprising Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, and wherein the intra-granular portion comprises Famotidine or salt or hydrates or solvates thereof and a pharmaceutically acceptable excipient, and the extra-granular portion comprises Fexofenadine or salt or hydrates or solvates thereof, Melatonin or salt or hydrates or solvates thereof and a pharmaceutically acceptable excipient.

In yet another embodiment, there is disclosed a fixed dose pharmaceutical composition comprising Fexofenadine or salt or hydrates or solvates thereof, Famotidine or salt or hydrates or solvates thereof and Melatonin or salt or hydrates or solvates thereof, wherein the composition comprises an intra-granular portion and an extra-granular portion, and wherein the intra-granular portion comprises Melatonin or salt or hydrates or solvates thereof and a pharmaceutically acceptable excipients and the extra-granular portion comprises Fexofenadine or salt or hydrates or solvates thereof and Famotidine or salt or hydrates or solvates thereof and a pharmaceutically acceptable excipient.

In one embodiment, the portions are compressed together to obtain a tablet dosage form, optionally coated with a seal coat. In an embodiment, the seal coat is an aqueous seal coat.

All types of the tablets mentioned hereinabove may be without a coating or may have one or more coatings, in particular, film-coatings. A film coating may be useful in limiting photolytic degradation and/or in reducing the degradation of moisture sensitive materials.

In one embodiment, the tablet may be coated to delay disintegration and/or absorption and thereby provide sustained action over a longer period. The non-limiting examples of film coating includes glyceryl monostearate or glyceryl distearate, polyvinyl alcohol based coatings, hydroxyethylcellulose, hydroxypropylmethylcellulose, sodium carboxymethylcellulose, polyethelene glycol 4000 and cellulose acetate phthalate film coating.

The compositions realized in accordance with embodiments of the present disclosure can find utility in treatment of urticaria, atopic dermatitis, pruritus and the like allergic reactions and/or dermatological diseases/conditions. Without wishing to be bound by the theory, it is believed that the combination of Fexofenadine or salt or hydrates or solvates thereof, Famotidine or salt or hydrates or solvates thereof, and Melatonin or salt or hydrates or solvates thereof, in particular, the fixed dose compositions disclosed herein, afford treatment of urticaria, atopic dermatitis, pruritus and the likes by decreasing the intensity of itching and by decreasing the surface area of itching, while reducing the night-time itching and sleep latency period. Several cohort studies have confirmed that more than half of the patients suffering from urticaria, atopic dermatitis and like chronic dermatological diseases/conditions suffer from moderate-to-significant sleep disruption, decreased confidence, depression and anxiousness. The sleep inducing potential of the compositions of the present disclosure can afford dramatic improvement in the quality of sleep of the patients of such chronic dermatological diseases while relieving or at least decreasing the intensity of itching and surface area of itching. Preliminary in-vitro study established the efficacy of the presently disclosed compositions. It could also be noted that the components of the compositions realized in accordance with embodiments of the present disclosure exhibit high degree of functional reciprocity by targeting different pathways and consequently, afford unique treatment options for urticaria, atopic dermatitis, pruritus and the like acute and/or chronic allergic reactions and/or dermatological diseases/conditions. Without wishing to be bound by the theory, it is also believed that the compositions realized in accordance with embodiments of the present disclosure exhibit high degree of functional reciprocity, wherein one or more active agents of the composition aids in retarding metabolism of the other active agent(s), and consequently, may reduce the dosage requirements and/or may aid in affording a prolonged action/efficacy. Specifically, it is believed that Famotidine undergoes metabolism (about 25-30%) through Cytochrome P450 system (CYP1A2) and excretion thereof may be decreased when combined with Melatonin. Similarly, Fexofenadine undergoes metabolism through Cytochrome P450 system (CYP3A2) and metabolism thereof may be decreased when combined with Famotidine. Similarly, Melatonin undergoes metabolism through Cytochrome P450 system (CYP1A1) and metabolism thereof may be decreased when combined with Fexofenadine.

Accordingly, an embodiment of the present disclosure provides a method of treatment of a dermatological condition/disorder/disease in a subject, said method comprising administering to a subject in need thereof an effective amount of a pharmaceutical composition comprising Fexofenadine or salt or hydrates or solvates thereof, Famotidine or salt or hydrates or solvates thereof and Melatonin or salt or hydrates or solvates thereof. In an embodiment, the dermatological condition includes any or a combination of: urticaria, dermatitis, atopic dermatitis and pruritus.

Another embodiment of the present disclosure provides a method of treatment of an allergic reaction in a subject, said method comprising administering to a subject in need thereof an effective amount of a pharmaceutical composition comprising Fexofenadine or salt or hydrates or solvates thereof, Famotidine or salt or hydrates or solvates thereof, and Melatonin or salt or hydrates or solvates thereof. In an embodiment, the allergic reaction is associated with or marked by elevated levels of allergen-specific IgE and/or T helper 2 (TH2) cells.

Further embodiment of the present disclosure provides a method of treatment of a sleep disorder/disease in a subject, said method comprising administering to a subject in need thereof an effective amount of a pharmaceutical composition comprising Fexofenadine or salt or hydrates or solvates thereof, Famotidine or salt or hydrates or solvates thereof, and Melatonin or salt or hydrates or solvates thereof.

Further embodiment of the present disclosure provides a pharmaceutical composition for use in treatment of a dermatological condition/disorder/disease, said composition comprising Fexofenadine or salt or hydrates or solvates thereof, Famotidine or salt or hydrates or solvates thereof, and Melatonin or salt or hydrates or solvates thereof. In an embodiment, the dermatological condition is any or a combination of: urticaria, dermatitis, atopic dermatitis and pruritus.

Further embodiment of the present disclosure provides a pharmaceutical composition for use in treatment of an allergic reaction, said composition comprising Fexofenadine or salt or hydrates or solvates thereof, Famotidine or salt or hydrates or solvates thereof, and Melatonin or salt or hydrates or solvates thereof. In an embodiment, the allergic reaction is associated with or marked by elevated levels of allergen-specific IgE and/or T helper 2 (TH2) cells.

Further embodiment of the present disclosure provides a pharmaceutical composition for use in treatment of a sleep disorder, said composition comprising Fexofenadine or salt or hydrates or solvates thereof, Famotidine or salt or hydrates or solvates thereof, and Melatonin or salt or hydrates or solvates thereof.

Yet another embodiment of the present disclosure provides use of a pharmaceutical composition for manufacture of a medicament for treatment of a dermatological condition/disorder/disease, said composition comprising Fexofenadine or salt or hydrates or solvates thereof, Famotidine or salt or hydrates or solvates thereof, and Melatonin or salt or hydrates or solvates thereof. In an embodiment, the dermatological condition includes any or a combination of: urticaria, dermatitis, atopic dermatitis and pruritus.

Yet another embodiment of the present disclosure provides use of a pharmaceutical composition for manufacture of a medicament for treatment of an allergic reaction, said composition comprising Fexofenadine or salt or hydrates or solvates thereof, Famotidine or salt or hydrates or solvates thereof, and Melatonin or salt or hydrates or solvates thereof. In an embodiment, the allergic reaction is associated with or marked by elevated levels of allergen-specific IgE and/or T helper 2 (TH2) cells.

Yet another embodiment of the present disclosure provides use of a pharmaceutical composition for manufacture of a medicament for treatment of a sleep disorder, said composition comprising Fexofenadine or salt or hydrates or solvates thereof, Famotidine or salt or hydrates or solvates thereof, and Melatonin or salt or hydrates or solvates thereof.

Further embodiment of the present disclosure provides a pharmaceutical composition for treatment of a dermatological condition/disorder/disease, said composition comprising Fexofenadine or salt or hydrates or solvates thereof, Famotidine or salt or hydrates or solvates thereof, and Melatonin or salt or hydrates or solvates thereof. In an embodiment, the dermatological condition/disorder/disease includes any or a combination of: urticaria, dermatitis, atopic dermatitis and pruritus.

Further embodiment of the present disclosure provides a pharmaceutical composition for treatment of an allergic reaction, said composition comprising Fexofenadine or salt or hydrates or solvates thereof, Famotidine or salt or hydrates or solvates thereof, and Melatonin or salt or hydrates or solvates thereof. In an embodiment, the allergic reaction is associated with or marked by elevated levels of allergen-specific IgE and/or T helper 2 (TH2) cells.

Further embodiment of the present disclosure provides a pharmaceutical composition for treatment of a sleep disorder, said composition comprising Fexofenadine or salt or hydrates or solvates thereof, Famotidine or salt or hydrates or solvates thereof, and Melatonin or salt or hydrates or solvates thereof.

The compositions of the present disclosure affords increased therapeutic effects, and reduced adverse effects, making these pharmaceutical compositions extremely effective therapeutics, especially in the treatment of acute or chronic allergic reactions, dermatological diseases/conditions and/or sleep disorders. Therapeutic levels of the combined drugs will vary from individual to individual and progression stage of disease. The combination medications in the appropriate amounts and intervals effective to treat urticaria, atopic dermatitis, pruritus, Covid-19 associated inflammatory complications such as Cytokines release etc., as well as gastrointestinal diseases such as GERD, gastritis, severe GERD and the like acute or chronic allergic reactions and/or dermatological diseases/conditions, gastrointestinal and sleep disorders may be monitored both clinically and chemically by the medical experts or trained physicians. The relevant formulation can eventually take the form of a combined pill given daily, a daily or weekly patch, a long-term injection, an implant, or a short-acting or long-acting form of medication.

Further, the patient may receive the specific dosage over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like.

The choice of appropriate dosages for the drugs used in combination therapy according to the present disclosure can be determined and optimized by the skilled artisan, e.g., by observation of the patient, including the patient's overall health, the response to the combination therapy, and the like. Optimization, for example, may be necessary if it is determined that a patient is not exhibiting the desired therapeutic effect or conversely, if the patient is experiencing undesirable or adverse side effects that are too many in number or are of a troublesome severity.

It is especially advantageous to formulate compositions of the present disclosure in unit dosage form for ease of administration and uniformity of dosage. The specifications of the dosage unit forms of the present disclosure are dependent on the unique characteristics of the composition and the particular therapeutic effect to be achieved. Dosages can further be determined by reference to the usual dose and manner of administration of the ingredients. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995).

### EXAMPLES

### Synthesis of Fatty Acid Derivatives

### Scheme I: Synthesis of 4-((1,3-bis(octanoyloxy)propan-2-yl)oxy)-4-oxobutanoic acid (Diglyceryl Capryl Succinate)

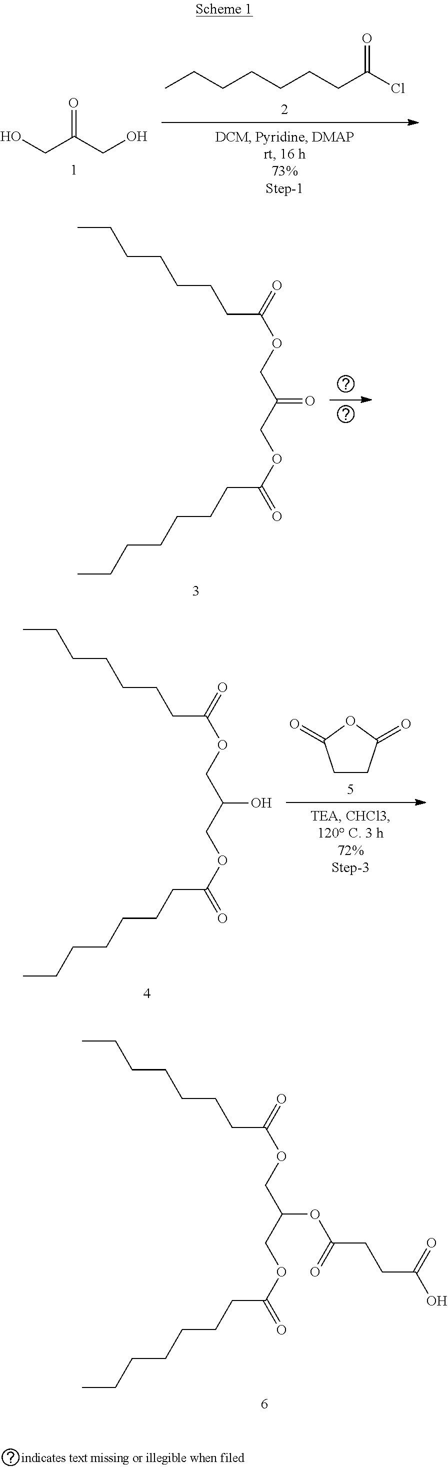

Step 1: Synthesis of 2-oxopropane-1,3-diyl dioctanoate (3): To an ice cold solution of 1,3-dihydroxypropan-2-one (1, 25.0 g, 0.277 mol) in dichloromethane (500 mL) was added 4-dimethylaminopyridine (10.17 g, 0.083 mol) and pyridine (49.2 mL, 0.610 mol) and stirred for next 5 min. To the above mixture octanoyl chloride (2, 105.4 mL, 0.610 mol) was added dropwise at 0° C., and the reaction mixture was stirred at room temperature for 16 h. After completion, reaction mixture was filtered; the solid was washed with dichloromethane (100 mL), filtrate was washed with brine (200 mL), saturated solution of sodium bicarbonate (200 mL) and 0.1 N HCl solution (100 mL). Organic layer was separated and dried over anhydrous sodium sulfate and solvent was removed under reduced pressure to get crude. The crude was purified by silica gel (100-200 mesh) column chromatography eluting with 10% ethyl acetate in hexanes to afford the desired product as white solid. Yield: 70.0 g, 73%. MS (ESI) m/z 343.19[M+1]⁺; ¹H NMR (400 MHz, DMSO-d6); δ 4.84 (s, 4H), 2.37 (t, J=7.2 Hz, 4H), 1.45-1.62 (m, 4H), 1.15-1.35 (m, 16H), 0.78-0.92 (m, 6H).

Step 2: Synthesis of 2-hydroxypropane-1,3-diyl dioctanoate (4): To an ice cold solution of 2-oxopropane-1,3-diyl dioctanoate (3, 70.0 g, 0.204 mol) in THF (1000 mL) was added drop wise acetic acid (15 mL), followed by the portion wise addition of sodium cyanoborohydride (15.43 g, 0.245 mol). The reaction mixture was stirred at room temperature for 16 h. After completion, reaction mixture was diluted with water (400 mL) and extracted with ethyl acetate (3×200 mL). The organic layer was separated, dried over anhydrous sodium sulfate and solvent was removed under reduced pressure. The crude thus obtained was purified by silica gel (100-200 mesh) column chromatography eluting with 12 to 15% ethyl acetate in hexanes to afford the desired product 4 as yellow liquid. Yield: 50.0 g, 71%. MS (ESI)-m/z 345.29[M+1]⁺; ¹H NMR (400 MHz, DMSO-d6); δ 5.25 (d, J=5.2 Hz, 1H), 3.92-4.03 (m, 4H), 3.81-3.90 (m, 1H), 2.29 (t, J=7.6 Hz, 4H), 1.45-1.59 (m, 4H), 1.12-1.35 (m, 16H), 0.85 (t, J=6.8 Hz, 6H).

Step 3: Synthesis of 4-((1,3-bis(octanoyloxy)propan-2-yl)oxy)-4-oxobutanoic acid (6): To a solution of 2-hydroxypropane-1,3-diyl dioctanoate (4, 50.0 g, 0.145 mol) in chloroform (200 mL), dihydrofuran-2,5-dione (5, 17.44 g, 0.174 mol) and triethylamine (30.0 mL, 0.218 mol) were added at room temperature. The reaction mixture was stirred at 120° C. for 3 h. After completion, reaction mixture was diluted with water (200 mL) and extracted with 1,2 dichloromethane (3×200 mL). The organic layer was separated, dried over anhydrous sodium sulfate and concentrated under reduced pressure. The crude thus obtained was purified by silica gel (100-200 mesh) column chromatography eluting with 10 to 15% ethyl acetate in hexanes to affored the desired product 6 as white solid. Yield: 47.0 g, 72%. MS (ESI)-m/z 443.2[M−1]; ¹H NMR (400 MHz, DMSO-d6): δ 12.22 (s, 1H), 5.12-5.22 (m, 1H), 4.18-4.25 (m, 2H), 4.09-4.17 (m, 2H), 2.42-2.50 (m, 4H), 2.29 (t, J=7.24 Hz, 4H), 1.44-1.55 (m, 4H), 1.15-1.31 (m, 16H), 0.79-0.90 (m, 6H).

### Scheme II: Synthesis of 4-((1,3-bis(dodecanoyloxy)propan-2-yl)oxy)-4-oxobutanoic acid (Diglyceryl Lauryl Succinate)

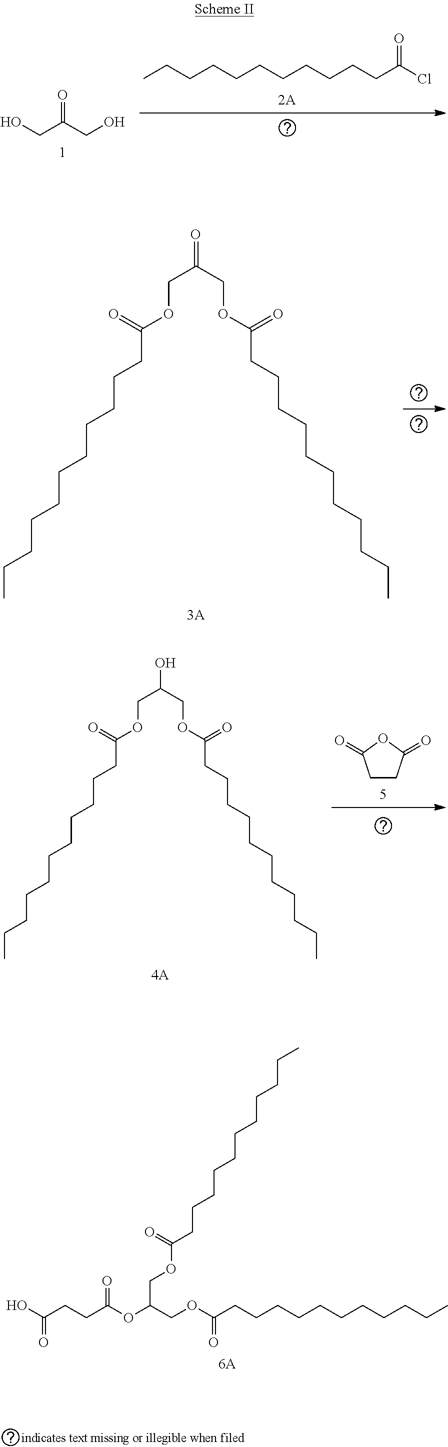 [SMILES: CCCCCCCCCCCC(=O)Cl ; O=C(CO)CO ; CCCCCCCCCCCC(=O)OCC(=O)COC(=O)CCCCCCCCCCC ; CCCCCCCCCCCC(=O)OCC(O)COC(=O)CCCCCCCCCCC ; O=C1CCC(=O)O1 ; CCCCCCCCCCCC(=O)OCC(COC(=O)CCCCCCCCCCC)OC(=O)CCC(=O)O]

Step 1: Synthesis of 2-oxopropane-1,3-diyl didodecanoate (3A): To an ice cold solution of 1,3-dihydroxypropan-2-one (1, 30.0 g, 0.33 mol) in dichloromethane (500 mL) was added 4-dimethylaminopyridine (20.30 g, 0.167 mol) and pyridine (107 mL, 0.1.332 mol) and stirred for next 5 min. To the above mixture dodecanoyl chloride 2A (218.50 g, 1.167 mol) was added dropwise at 0° C. and the reaction mixture was stirred at room temperature for 16 h. After completion, reaction mixture was filtered; the solid was washed with dichloromethane (100 mL), filtrate was washed with brine (200 mL), saturated solution of sodium bicarbonate (200 mL) and 0.1 N HCl solution (100 mL). Organic layer was separated and dried over anhydrous sodium sulfate and solvent was removed under reduced pressure to get crude. The crude was triturated with diethyl ether to afford the desired product 3A as white solid. Yield: 78 g, 51%. MS (ESI) m/z 455.37[M+1]⁺; ¹H NMR (400 MHz, DMSO-d6)−δ 4.74 (s, 4H), 2.43 (m, 4H), 1.64 (m, 4H), 1.55-1.25 (m, 32H), 0.87 (m, 6H).

Step 2: Synthesis of 2-hydroxypropane-1,3-diyl didodecanoate (4A): To an ice cold solution of 2-oxopropane-1,3-diyl didodecanoate 3A (75.0 g, 0.165 mol) in THF (1000 mL) was added drop wise acetic acid (15 mL) followed by the portion wise addition of sodium cyanoborohydride (12.41 g, 0.198 mol). The reaction mixture was stirred at room temperature for 16 h. After completion, reaction mixture was diluted with water (400 mL) and extracted with ethyl acetate (3×200 mL). The organic layer was separated, dried over anhydrous sodium sulfate and solvent was removed under reduced pressure. The crude was triturated with diethyl ether to afford the desired product 4A as white solid. Yield: 60.0 g, 80%. MS (ESI); m/z 457.48[M+1]⁺; ¹H NMR (400 MHz, DMSO-d6)−δ 5.26 (d, J=5.2 Hz, 1H), 3.92-3.98 (m, 4H), 2.28 (m, 4H), 1.50 (m, 4H), 1.23 (m, 33H), 0.83 (m, 6H).

Step 3: Synthesis of 4-((1,3-bis(dodecanoyloxy)propan-2-yl)oxy)-4-oxobutanoic acid (6A): To a solution of 2-hydroxypropane-1,3-diyl didodecanoate 4A (40.0 g, 0.087 mol) in chloroform (200 mL), dihydrofuran-2,5-dione 5 (10.50 g, 0.105 mol) and triethylamine (18.50 mL, 0.131 mol) were added at room temperature. The reaction mixture was stirred at 120° C. for 3 h. After completion, reaction mixture was diluted with water (200 mL) and extracted with 1,2 dichloromethane (3×200 mL). The organic layer was separated, dried over anhydrous sodium sulfate and concentrated under reduced pressure. The crude thus obtained was purified by silica gel (100-200 mesh) column chromatography eluting with 25 to 30% ethyl acetate in hexanes to afford the desired product 6A as white solid. Yield: 20.0 g, 41%. MS (ESI) m/z 555.40[M−1]; ¹H NMR (400 MHz, DMSO-d6) δ 12.30 (s, 1H), 5.17 (m, 1H), 4.18-4.25 (m, 4H), 2.50-2.47 (m, 8H), 1.23-1.25 (m, 36H), 0.83 (m, 6H).

### Scheme III: Synthesis of (E)-4-((1,3-bis(dodecanoyloxy)propan-2-yl)oxy)-4-oxobut-2-enoic acid (Diglyceryl Lauryl Fumarate)

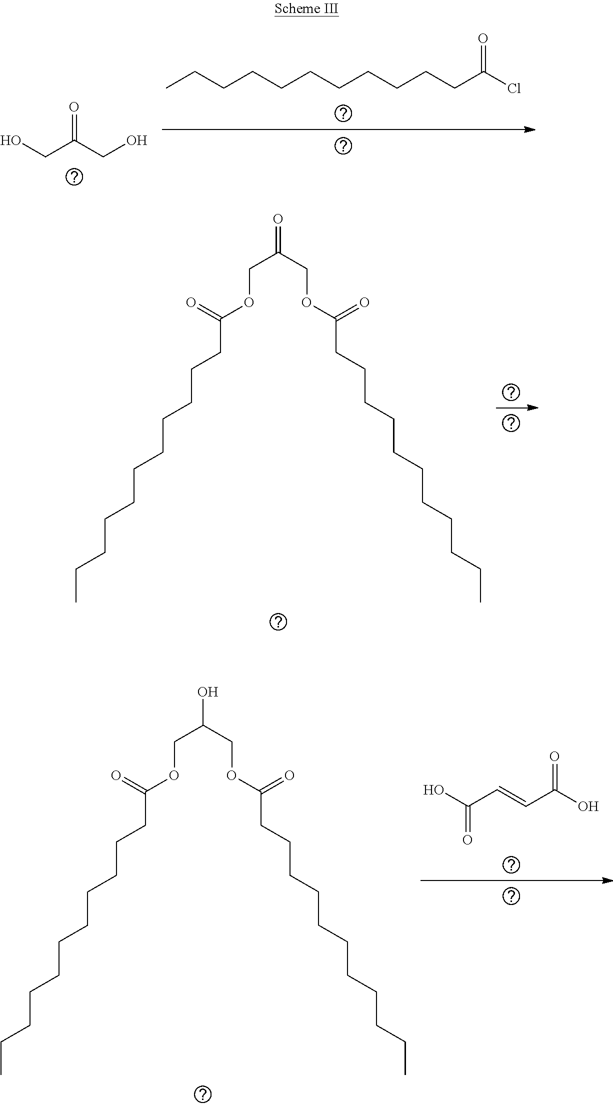 [SMILES: CCCCCCCCCCCC(=O)Cl ; O=C(CO)CO ; CCCCCCCCCCCC(=O)OCC(=O)COC(=O)CCCCCCCCCCC ; CCCCCCCCCCCC(=O)OCC(O)COC(=O)CCCCCCCCCCC ; O=C(O)/C=C/C(=O)O]

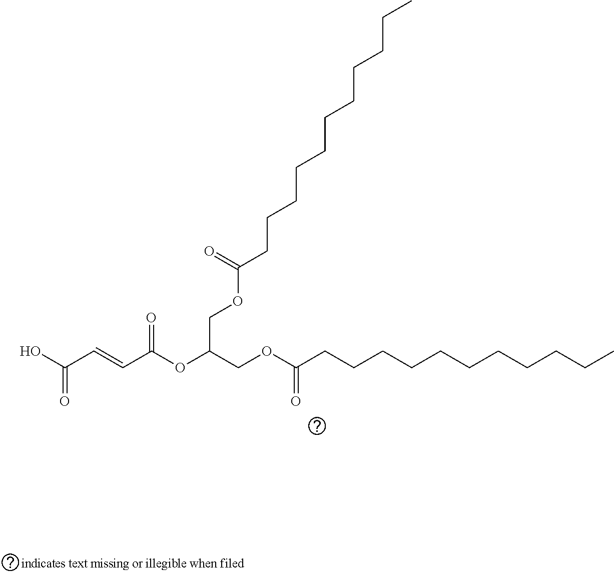 [SMILES: CCCCCCCCCCCC(=O)OCC(COC(=O)CCCCCCCCCCC)OC(=O)/C=C/C(=O)O]

Step 1—Synthesis of 2-oxopropane-1,3-diyl didodecanoate (3*): To an ice cold solution of 1,3-dihydroxypropan-2-one (1, 30.0 g, 0.33 mol) in dichloromethane (500 mL) was added 4-dimethylaminopyridine (20.30 g, 0.167 mol) and pyridine (107 mL, 0.1.332 mol) and stirred for next 5 min. To the above reaction mixture dodecanoyl chloride 2 (218.50 g, 1.167 mol) was added dropwise at 0° C. and the reaction mixture was stirred at room temperature for 16 h. After completion, reaction mixture was filtered, the solid was washed with dichloromethane (100 mL), filtrate was washed with brine (200 mL), saturated solution of sodium bicarbonate (200 mL) and 0.1 N HCl solution (100 mL). The organic layer was separated, dried over anhydrous sodium sulfate and solvent was removed under reduced pressure to get crude. The crude was triturated with diethyl ether to afford the desired product 3* as white solid. Yield: 78 g, 51%. MS (ESI) m/z 455.37[M+1]⁺; 1H NMR (400 MHz, DMSO-d6): δ 4.74 (s, 4H), 2.43 (m, 4H), 1.64 (m, 4H), 1.55-1.25 (m, 32H), 0.87 (m, 6H).

Step-2: Synthesis of 2-hydroxypropane-1,3-diyl didodecanoate (4*): To an ice cold solution of 2-oxopropane-1,3-diyl didodecanoate 3 (75.0 g, 0.165 mol) in THF (1000 mL) was added drop wise acetic acid (15 mL) followed by the portion wise addition of sodium cyanoborohydride (12.41 g, 0.198 mol). The reaction mixture was stirred at room temperature for 16 h. After completion, reaction mixture was diluted with water (400 mL) and extracted with ethyl acetate (3×200 mL). The organic layer was separated, dried over anhydrous sodium sulfate and solvent was removed under reduced pressure. The crude was triturated with diethyl ether to afford the desired product 4* as white solid. Yield: 60.0 g, 80%. MS (ESI) m/z 457.48[M+1]⁺; 1H NMR (400 MHz, DMSO-d6): δ 5.26 (d, J=5.2 Hz, 1H), 3.92-3.98 (m, 4H), 2.28 (m, 4H), 1.50 (m, 4H), 1.23 (m, 33H) and 0.83 (m, 6H).

Step-3: Synthesis of (E)-4-((1,3-bis(dodecanoyloxy)propan-2-yl)oxy)-4-oxobut-2-enoic acid (6*): To an ice-cold solution of 2-hydroxypropane-1,3-diyl didodecanoate 4 (10.0 g, 21.91 mmol) in THF (170 mL) was added fumaric acid 5 (2.54 g, 21.91 mmol), benzoyl chloride (2.5 mL, 21.91 mmol) and DMAP (0.67 g, 5.477 mmol). The resulting mixture was stirred at RT for 16 h. After completion of reaction (TLC monitoring), reaction mixture was concentrated under reduced pressure. The crude was diluted with water (200 mL), adjust pH ˜2-3 using 1N—HCl and extracted with 1,2 dichloromethane (3×200 mL). The organic layer was separated, dried over anhydrous sodium sulfate and concentrated under reduced pressure. The crude thus obtained was purified by silica gel (100-200 mesh) column chromatography eluting with 80% ethyl acetate in hexanes to afford the desired product 6* as white solid. Yield: 400 mg, 3.30% (un-optimized yield). LC-MS: m/z 553.64[M−1]; 97.27% purity. 1H NMR (400 MHz, DMSO-d6): δ 13.26 (br s, 1H), 5.17 (d, J=15.8 Hz, 2H), 5.29 (m, 1H), 4.30-4.33 (m, 2H), 4.19-4.23 (m, 2H), 2.28 (m, 4H), 1.48 (m, 4H), 1.22 (m, 32H) and 0.83 (m, 6H).

### Non-Limiting Examplary Compositions

Batch ACGCP300102001A

TABLE 1
Composition for Fexofenadine, Famotidine and
Melatonin immediate release tablets

| Ingredients | mg/Unit | % w/w |
|---|---|---|
| Intragranular | | |
| Famotidine | 40.00 | 13.3 |
| Fexofenadine HCl | 60.00 | 20.0 |
| Melatonin | 4.00 | 1.3 |
| Microcrystalline | 48.00 | 16.0 |
| cellulose | | |
| Heavy magnesium oxide | 25.00 | 8.3 |
| Calcium carbonate | 25.00 | 8.3 |
| L-Carnosine | 10.00 | 3.3 |
| Diglyceryl lauryl | 10.00 | 3.3 |
| fumarate | | |
| Croscarmellose sodium | 14.00 | 4.7 |
| Binder solution | | |
| Hypromellose | 15.00 | 5.0 |
| Simethicone | 25.00 | 8.3 |
| Water | q. s | — |
| Extra-granular | | |
| Croscarmellose sodium | 10.00 | 3.3 |
| Colloidal Silicon Dioxide | 7.00 | 2.3 |
| Zinc stearate | 7.00 | 2.3 |
| Average tablet weight | 300.00 | 100.00 |

### Method of Preparation of Fexofenadine, Famotidine and Melatonin Tablets

### I. Granulation

i. Step 1—Dispensing: Dispensed all the required raw materials using a calibrated weighing balance.
ii. Step 2—Sifting: Fexofenadine (or its HCl salt), Famotidine, Melatonin, Microcrystalline cellulose, Magnesium oxide, Calcium carbonate, L-Carnosine, Diglyceryl lauryl fumarate and Croscarmellose sodium were sifted through ASTM #30 sieve.
iii. Step 3—Dry mixing: The sifted mixture was dry mixed in Rapid Mixer Granulator for 10 minutes at 50 RPM.
iv. Step 4—Binder solution preparation: Hypromellose 5 cps was dissolved in water and mixed well followed by the addition of Simethicone.
v. Step 5—Wet Mixing: The dry mix obtained in step 3 was granulated using binder solution prepared in step 4 to obtain granules.
vi. Step 6—Drying: Granules obtained in step 5 were dried at an inlet temperature of NMT 60° C. and product temperature of NMT 45° C. until LOD of NMT 3.5% is obtained.
vii. Step 7—Sizing: Dried granules were passed through ASTM sieve no. 30.
viii. Step 8—Blending: Granules of step 7 were blended with extra-granular materials (passed through ASTM 40 sieve) i.e., Croscarmellose sodium and colloidal silicon dioxide for 10 min at 15 RPM.
ix. Step 9—Lubrication: Blend obtained in step 8 was lubricated with #60 passed zinc stearate for 5 min at 15 RPM.

### II. Compression:

Lubricated blend obtained in step 9 was compressed. Compression parameters are provided in Table 2 below:

TABLE 2
Compression parameters

| Parameter | Observations |
|---|---|
| Tooling | 10.6 × 5.0 mm oval shape punches |
| Average tablet weight | 300.0 |
| (mg) | |
| Tablet thickness (mm) | 5.30-5.40 |
| Tablet hardness (kP) | 10-12 |
| Disintegration time (min) | 2-3 |
| Remarks | No sticking was observed |

### III. Packaging:

The obtained tablets were packed in 60 cc HDPE bottle along with one 2 g silica gel bag, induction sealed and closed with 33 mm CR closure.

### IV. Physicochemical Properties:

Stability and release parameters of the tablets were tested, results whereof are provided in Table 3 below.

TABLE 3
Physicochemical properties of tablets

| | | |
|---|---|---|
| Test Parameters | Acceptance criteria | Initial |

Assay (%)

| | | |
|---|---|---|
| Famotidine | 90.0-110.0% | 100.5 |
| Fexofenadine HCl | 90.0-110.0% | 99.8 |
| Melatonin | 90.0-110.0% | 99.8 |
| Water content (% w/w) | NMT 7.0 | |

Related Substances (%)

| | | |
|---|---|---|
| Famotidine IMP-C | NMT 0.5 | 0.00 |
| Famotidine IMP-D | NMT 0.5 | 0.02 |
| Famotidine IMP-E | NMT 0.3 | 0.04 |
| Famotidine IMP-F | NMT 0.5 | 0.00 |
| Famotidine IMP-I | NMT 1.0 | ND |
| Fexofenadine IMP-A | NMT 0.2 | 0.05 |
| Melatonin IMP-A | NMT 0.2 | ND |
| Any other highest unknown | NMT 0.2 | 0.05 |
| impurity | | |
| Total Impurities | NMT 3.0 | 0.55 |

Dissolution Method: Appts: USP II, Speed: 50 RPM, Volume: 900 mL

| | 0.001N HCl | | | pH 4.5 Phosphate buffer | | | pH 6.8 Phosphate buffer | | |
|---|---|---|---|---|---|---|---|---|---|
| Time | Famotidine | Fexofenadine | Melatonin | Famotidine | Fexofenadine | Melatonin | Famotidine | Fexofenadine | Melatonin |
| (min) | Average | Average | Average | Average | Average | Average | Average | Average | Average |
| 5 | 30 | 33 | 35 | 38 | 30 | 46 | 25 | 35 | 38 |
| 10 | 58 | 63 | 66 | 76 | 67 | 90 | 55 | 74 | 80 |
| 20 | 82 | 91 | 96 | 89 | 86 | 98 | 73 | 95 | 99 |
| 30 | 88 | 97 | 100 | 93 | 90 | 100 | 79 | 97 | 100 |
| 45 | 92 | 99 | 102 | 96 | 93 | 101 | 83 | 99 | 101 |
| 60 | 94 | 100 | 102 | 98 | 94 | 102 | 87 | 100 | 101 |

Batch ACGCP300102002A

Tablets were prepared using the composition as provided in Table 4 below:

TABLE 4
Composition for Fexofenadine, Famotidine and Melatonin Tablets

| Ingredients | mg/Unit | % w/w |
|---|---|---|
| Famotidine | 40.00 | 13.3 |
| Fexofenadine | 60.00 | 20.0 |
| Melatonin | 4.00 | 1.3 |
| MCC | 63.50 | 21.2 |
| Heavy magnesium oxide | 25.00 | 8.3 |
| Calcium carbonate | 25.00 | 8.3 |
| L-Camosine | 10.00 | 3.3 |
| HPMC 5 cps | 15.00 | 5.0 |
| Simethicone emulsion | 25.00 | 8.3 |
| Croscarmellose sodium | 24.00 | 8.0 |
| Colloidal Silicon Dioxide | 1.50 | 0.5 |
| Zinc stearate | 7.00 | 2.3 |
| Water | QS | |
| Avg tablet weight | 300.00 | 100 |

Tablet mass was directly subjected to compression. Compression parameters are provided in Table 5 below:

TABLE 5
Compression parameters

| Parameter | Observations |
|---|---|
| Tooling | 10.6 × 5.0 mm oval shape punches. |
| Average tablet weight | 300.0 |
| (mg) | |
| Tablet thickness (mm) | 5.20-5.40 |
| Tablet hardness (kP) | 9-11 |
| Disintegration time (min) | 9-12 |
| Remarks | No sticking was observed |

The obtained tablets were packed in 60 cc HDPE bottle along with one 2 g silica gel bag, induction sealed and closed with 33 mm CR closure. Stability and release parameters of the tablets were tested, results whereof are provided in Table 6 below.

TABLE 6
Physicochemical properties of tablets

| | | | | | |
|---|---|---|---|---|---|
| | | 40° C./75% | 40° C./75% | 40° C./75% | 25° C./60% |
| Test Parameters | Initial | RH-1M | RH-2M | RH-3M | RH-3M |

Assay (%)

| | | | | | |
|---|---|---|---|---|---|
| Famotidine | 103.3 | 105.4 | 104.3 | 103.6 | 105.2 |
| Fexofenadine | 100.5 | 100.4 | 100.9 | 102 | 101.5 |
| Melatonin | 100.1 | 99.9 | 100.7 | 104.5 | 102.6 |
| Water content | 5.03 | 6.15 | 6.36 | 4.87 | 4.97 |

Related Substances (%)

| | | | | | |
|---|---|---|---|---|---|
| Famotidine IMP-A | ND | ND | ND | ND | ND |
| Famotidine IMP-B | 0.04 | 0.04 | 0.04 | 0.03 | 0.04 |
| Famotidine IMP-C | 0.00 | 0.00 | 0.00 | 0.05 | 0.09 |
| Famotidine IMP-D | 0.02 | 0.03 | 0.03 | 0.04 | 0.05 |
| Famotidine IMP-E | 0.04 | 0.05 | 0.05 | 0.04 | 0.07 |
| Famotidine IMP-F | 0.00 | 0.00 | 0.00 | 0.01 | 0.01 |
| Famotidine IMP-G | 0.08 | 0.09 | 0.09 | 0.08 | 0.08 |
| Famotidine IMP-H | 0.08 | 0.12 | 0.09 | 0.02 | 0.04 |
| Famotidine IMP-I | 0.02 | 0.03 | 0.04 | 0.08 | 0.02 |
| Famotidine IMP-J | ND | ND | ND | ND | ND |
| Fexofenadine IMP-A | 0.05 | 0.08 | 0.09 | 0.13 | 0.10 |
| Melatonin IMP-A | ND | ND | ND | ND | ND |
| Any ukn highest of | 0.02 | 0.02 | 0.03 | 0.04 | 0.06 |
| Famo @1.284 | | | | | |
| Any ukn highest of Fexo | 0.05 | 0.05 | 0.05 | 0.01 | 0.01 |
| @ 1.241 | | | | | |
| Any ukn highest of | 0.04 | 0.04 | 0.04 | 0.04 | 0.04 |
| Mela @0.714 | | | | | |
| Total Impurities | 0.60 | 0.74 | 0.74 | 0.73 | 0.75 |

Dissolution Methodology: Medium: 0.001N HCl, Speed: 50 RPM, Volume: 900 mL, Appts: USP II

| | | | | | |
|---|---|---|---|---|---|
| | % drug | | | | |
| Time (min) | release | % drug release | % drug release | % drug release | % drug release |

Famotidine

| | | | | | |
|---|---|---|---|---|---|
| 5 | 27 | 24 | 21 | 26 | 26 |
| 10 | 57 | 51 | 42 | 50 | 51 |
| 20 | 83 | 79 | 71 | 77 | 79 |
| 30 | 89 | 90 | 82 | 88 | 87 |
| 45 | 93 | 94 | 87 | 92 | 92 |
| 60 | 95 | 99 | 91 | 94 | 94 |

Fexofenadine

| | | | | | |
|---|---|---|---|---|---|
| 5 | 32 | 24 | 22 | 26 | 26 |
| 10 | 65 | 54 | 45 | 49 | 52 |
| 20 | 93 | 88 | 79 | 80 | 85 |
| 30 | 98 | 99 | 93 | 94 | 92 |
| 45 | 100 | 100 | 97 | 98 | 95 |
| 60 | 101 | 103 | 99 | 100 | 96 |

Melatonin

| | | | | | |
|---|---|---|---|---|---|
| 5 | 28 | 23 | 22 | 25 | 27 |
| 10 | 60 | 54 | 43 | 48 | 54 |
| 20 | 94 | 87 | 78 | 79 | 86 |
| 30 | 100 | 99 | 92 | 94 | 93 |
| 45 | 102 | 95 | 97 | 97 | 96 |
| 60 | 103 | 103 | 100 | 99 | 98 |

Batch ACGCP300102004A

Coated tablets were prepared using the composition as provided in Table 7 below:

TABLE 7
Composition for Fexofenadine, Famotidine and Melatonin coated tablets

| Ingredients | mg/Unit | % w/w |
|---|---|---|
| Intragranular | | |
| Famotidine | 40.00 | 13.3 |
| Fexofenadine HCl | 60.00 | 20.0 |
| Melatonin | 4.00 | 1.3 |
| Microcrystalline cellulose | 58.00 | 16.0 |
| Heavy magnesium oxide | 25.00 | 8.3 |
| Calcium carbonate | 25.00 | 8.3 |
| L-Carnosine | 10.00 | 3.3 |
| Diglyceryl lauryl fumarate | 10.00 | 3.3 |
| Croscarmellose sodium | 14.00 | 4.7 |
| Binder solution | | |
| Hypromellose | 15.00 | 5.0 |
| Simethicone | 25.00 | 8.3 |
| Water | q. s | — |
| Extra-granular | | |
| Croscarmellose sodium | 10.00 | 3.3 |
| Colloidal Silicon Dioxide | 1.50 | 2.3 |
| Zinc stearate | 7.00 | 2.3 |
| Coating | | |
| Opadry II 85F565369 | 4.50 | 1.5% coating build-up |
| Water | q. s | — |

Tablets were prepared using the process as detailed for batch ACGCP300102001A. The compressed tablets were then subjected to coating. Compression parameters are provided in Table 8 below. Dissolution characteristics of the coated tablets were then studied and same are provided in Table 9 below.

TABLE 8
Compression parameters

| Parameter | Observations |
|---|---|
| Tooling | 10.6 × 5.0 mm oval shape punches. |
| Average tablet weight | 300 mg |
| (mg) | |
| Tablet thickness (mm) | 5.7-5.9 |
| Tablet hardness (kP) | 4-6 |
| Disintegration time (min) | 5-6 |
| Remarks | No sticking of tablets during compression |

TABLE 9
Dissolution characteristics
Initial

| | Famotidine | Fexofenadine | Melatonin |
|---|---|---|---|
| | Average | Average | Average |
| 5 | 39 | 38 | 70 |
| 10 | 88 | 86 | 96 |
| 20 | 105 | 94 | 97 |
| 30 | 107 | 96 | 97 |
| 45 | 108 | 96 | 97 |
| 60 | 109 | 96 | 97 |

Batch ACGCP300102005A

Coated tablets were prepared using the composition as provided in Table 10 below:

TABLE 10
Composition for Fexofenadine, Famotidine and Melatonin Coated tablets

| Ingredients | mg/Unit | % w/w |
|---|---|---|
| Intragranular | | |
| Famotidine | 40.00 | |
| Fexofenadine | 60.00 | 20.3 |
| Melatonin | 4.00 | 1.4 |
| MCC | 59.50 | 20.2 |
| Heavy magnesium oxide | 25.00 | 8.5 |
| Calcium carbonate | 25.00 | 8.5 |
| L-Carnosine | 10.00 | 3.4 |
| Croscarmellose sodium | 14.00 | 4.7 |
| Simethicone powder | 25.00 | 8.5 |
| Binder solution | | |
| HPMC 5 cps | 15.00 | 5.1 |
| Water | QS | — |
| Extragranular | | |
| Croscarmellose sodium | 10.00 | 3.4 |
| Colloidal Silicon Dioxide | 1.50 | 0.5 |
| Zinc stearate | 6.00 | 2.0 |
| Avg tablet weight | 295.00 | 100 |
| Coating | | |
| Opadry II 85F565369 | 9.00 | 3.0 |
| | | (3% coating build-up) |
| Water | q.s | — |
| Coated tablet weight | 309 | — |

Tablets were prepared using the process as detailed for batch ACGCP300102001A. The compressed tablets were then subjected to coating. Compression parameters are provided in Table 11 below. Characteristics of the coated tablets were then studied and same are provided in Table 12 below.

TABLE 11
Compression parameters

| Parameter | Observations |
|---|---|
| Tooling | 10.6 × 5.0 mm oval shape punches. |
| Average tablet weight (mg) | 309 mg |
| Tablet thickness (mm) | 5.7-5.9 |
| Tablet hardness (kP) | 4-6 |
| Disintegration time (min) | 5-6 |
| Remarks | No sticking of tablets during compression |

TABLE 12
Characteristics of coated tablets

| | 60 cc HDPE bottle/33 mm CR closure with one 2 g silica gel bag | | | | |
|---|---|---|---|---|---|
| Container Closure | | 40° C./75% | 40° C./75% | 40° C./75% | 25° C./60% |
| Test Parameters | Initial | RH-1M | RH-2M | RH-3M | RH-3M |

Assay (%)

| | | | | | |
|---|---|---|---|---|---|
| Famotidine | 97.4 | 96.9 | 96.3 | 98.6 | 97.1 |
| Fexofenadine | 97.0 | 97.1 | 96.6 | 97.7 | 96.9 |
| Melatonin | 95.2 | 95.7 | 94.9 | 96.8 | 95.7 |
| Water content | 4.29 | 4.4 | | | |

Related Substances (%)

| | | | | | |
|---|---|---|---|---|---|
| Famotidine IMP-A | ND | ND | ND | ND | ND |
| Famotidine IMP-B | 0.05 | 0.06 | 0.06 | 0.04 | 0.05 |
| Famotidine IMP-C | 0.04 | 0.08 | 0.03 | 0.09 | 0.06 |
| Famotidine IMP-D | 0.04 | 0.07 | 0.01 | 0.05 | 0.03 |
| Famotidine IMP-E | 0.08 | 0.20 | 0.06 | 0.06 | 0.06 |
| Famotidine IMP-F | 0.01 | 0.01 | 0.01 | 0.03 | 0.02 |
| Famotidine IMP-G | 0.07 | 0.07 | 0.07 | 0.07 | 0.07 |
| Famotidine IMP-H | 0.09 | 0.09 | 0.02 | 0.03 | 0.02 |
| Famotidine IMP-I | 0.02 | 0.05 | 0.03 | 0.02 | 0.02 |
| Famotidine IMP-J | ND | ND | ND | ND | ND |
| Fexofenadine IMP-A | 0.04 | 0.04 | 0.05 | 0.08 | 0.05 |
| Melatonin IMP-A | 0.01 | ND | ND | 0.01 | 0.01 |
| Any ukn highest of | 0.04 | 0.05 | 0.04 | 0.03 | 0.02 |
| Famo @1.284 | | | | | |
| Any ukn highest of Fexo | 0.07 | 0.05 | 0.05 | 0.01 | 0.01 |
| @ 1.241 | | | | | |
| Any ukn highest of | 0.14 | 0.18 | 0.16 | 0.07 | 0.05 |
| Mela @0.714 | | | | | |
| Total Impurities | 0.84 | 1.15 | 0.76 | 0.88 | 0.69 |

Dissolution Methodology: Medium: 0.001N HCl, Speed: 50 RPM, Volume: 900 mL, Appts: USP II

| | | | |
|---|---|---|---|
| Time (min) | % drug release | % drug release | % drug release |

Famotidine

| | | | |
|---|---|---|---|
| 5 | 36 | 41 | 37 |
| 10 | 77 | 81 | 76 |
| 20 | 90 | 94 | 94 |
| 30 | 92 | 97 | 96 |
| 45 | 94 | 98 | 98 |
| 60 | 95 | 100 | 99 |

Fexofenadine

| | | | |
|---|---|---|---|
| 5 | 34 | 35 | 37 |
| 10 | 77 | 76 | 76 |
| 20 | 93 | 93 | 83 |
| 30 | 95 | 95 | 97 |
| 45 | 96 | 96 | 97 |
| 60 | 97 | 97 | 97 |

Melatonin

| | | | |
|---|---|---|---|
| 5 | 42 | 43 | 39 |
| 10 | 88 | 87 | 81 |
| 20 | 95 | 94 | 94 |
| 30 | 96 | 95 | 96 |
| 45 | 96 | 95 | 97 |
| 60 | 96 | 96 | 97 |

Batch ACGCP300102006A

Coated tablets were prepared using the composition as provided in Table 13 below:

TABLE 13
Composition for Fexofenadine, Famotidine and Melatonin coated tablets

| Ingredients | mg/Unit | % w/w |
|---|---|---|
| Intragranular | | |
| Famotidine | 40.00 | 13.6 |
| Fexofenadine | 60.00 | 20.3 |
| Melatonin | 4.00 | 1.4 |
| MCC | 34.50 | 11.7 |
| Heavy magnesium oxide | 25.00 | 8.5 |
| Calcium carbonate | 25.00 | 8.5 |
| L-Carnosine | 10.00 | 3.4 |
| Croscarmellose sodium | 14.00 | 4.7 |
| Simethicone powder | 25.00 | 8.5 |
| Binder solution | | |
| HPMC 5 cps | 15.00 | 5.1 |
| Water | QS | |
| Extragranular | | |
| Pregelatinized starch | 30.00 | |
| Croscarmellose sodium | 10.00 | 3.4 |
| Colloidal Silicon Dioxide | 1.50 | 0.5 |
| Zinc stearate | 6.00 | 2.0 |
| Avg tablet weight | 300.00 | |
| Coating | | |
| Opadry II 85F565369 | 9.00 | 3% coating build-up |
| Water | q.s | |
| Coated tablet weight | 309 | |

Tablets were prepared using the process as detailed for batch ACGCP300102001A. The compressed tablets were then subjected to coating. Compression parameters are provided in Table 13 below. Characteristics of the coated tablets were then studied and same are provided in Table 14 below.

TABLE 13
Compression parameters

| Parameter | Observations |
|---|---|
| Tooling | 10.6 × 5.0 mm oval shape punches. |
| Average tablet weight (mg) | 300 mg |
| Tablet thickness (mm) | 5.7-5.9 |
| Tablet hardness (kP) | 4-6 |
| Disintegration time (min) | 5-6 |
| Remarks | No sticking of tablets during compression |

TABLE 14
Characteristics of the coated tablets

| Container Closure | | | | |
|---|---|---|---|---|
| 60 cc HDPE bottle/33 mm CR closure with one 2 g silica gel bag | | | | |
| Test Parameters | | | | |
| | 40° C./75% | 40° C./75% | 40° C./75% | 25° C./60% |
| Initial | RH − 1M | RH − 2M | RH − 3M | RH − 3M |

Assay (%)

| | | | | | |
|---|---|---|---|---|---|
| Famotidine | 98.3 | 99.3 | 99.8 | 100.8 | 100.5 |
| Fexofenadine | 95.4 | 96.8 | 96.3 | 97.4 | 97.0 |
| Melatonin | 95.4 | 96.6 | 97.6 | 97.2 | 96.9 |
| Water content | 4.36 | 4.39 | — | 5.83 | 5.61 |

Related Substances (%)

| | | | | | |
|---|---|---|---|---|---|
| Famotidine IMP-A | ND | ND | ND | ND | ND |
| Famotidine IMP-B | 0.06 | 0.05 | 0.06 | 0.05 | 0.05 |
| Famotidine IMP-C | 0.01 | 0.01 | 0.04 | 0.10 | 0.06 |
| Famotidine IMP-D | 0.05 | 0.03 | 0.04 | 0.04 | 0.04 |
| Famotidine IMP-E | 0.12 | 0.06 | 0.06 | 0.05 | 0.06 |
| Famotidine IMP-F | 0.01 | 0.01 | 0.01 | 0.03 | 0.02 |
| Famotidine IMP-G | 0.07 | 0.09 | 0.08 | 0.07 | 0.07 |
| Famotidine IMP-H | 0.09 | 0.09 | 0.02 | 0.03 | 0.03 |
| Famotidine IMP-I | 0.07 | 0.01 | 0.03 | 0.02 | 0.02 |
| Famotidine IMP-J | ND | ND | ND | ND | ND |
| Fexofenadine IMP-A | 0.05 | 0.07 | 0.11 | 0.12 | 0.07 |
| Melatonin IMP-A | 0.01 | ND | ND | 0.01 | 0.01 |
| Any ukn highest of | 0.04 | 0.03 | 0.04 | 0.03 | 0.03 |
| Famo @1.284 | | | | | |
| Any ukn highest of | 0.05 | 0.06 | 0.01 | 0.08 | 0.01 |
| Fexo @ 1.241 | | | | | |
| Any ukn highest of | 0.17 | 0.05 | 0.06 | 0.06 | 0.05 |
| Mela @0.714 | | | | | |
| Total Impurities | 1.01 | 0.76 | 0.73 | 0.89 | 0.75 |

Dissolution Methodology: Medium: 0.001N HCl, Speed: 50 RPM,
Volume: 900 mL, Appts: USP II

| | | | | | |
|---|---|---|---|---|---|
| | % drug | % drug | % drug | % drug | % drug |
| Time (min) | release | release | release | release | release |

Famotidine

| | | | | | |
|---|---|---|---|---|---|
| 5 | 38 | 40 | NA | 47 | 55 |
| 10 | 76 | 77 | | 79 | 85 |
| 20 | 85 | 88 | | 87 | 90 |
| 30 | 88 | 90 | | 89 | 93 |
| 45 | 91 | 91 | | 90 | 95 |
| 60 | 93 | 92 | | 91 | 96 |

Fexofenadine

| | | | | | |
|---|---|---|---|---|---|
| 5 | 40 | 34 | NA | 48 | 56 |
| 10 | 82 | 75 | | 81 | 87 |
| 20 | 92 | 93 | | 91 | 93 |
| 30 | 94 | 95 | | 94 | 94 |
| 45 | 95 | 96 | | 95 | 95 |
| 60 | 97 | 96 | | 94 | 95 |

Melatonin

| | | | | | |
|---|---|---|---|---|---|
| 5 | 38 | 44 | NA | 55 | 62 |
| 10 | 86 | 89 | | 92 | 95 |
| 20 | 94 | 96 | | 95 | 97 |
| 30 | 96 | 97 | | 97 | 98 |
| 45 | 96 | 97 | | 97 | 98 |
| 60 | 96 | 97 | | 96 | 99 |

Batch ACGCP300102005A

Coated tablets were prepared using the composition as provided in Table 15 below. In this batch, micronized Famotidine was used to understand effect thereof on characteristics of the coated tablets.

TABLE 15
Composition for Fexofenadine, Famotidine and Melatonin coated tablets

| Ingredients | mg/Unit | % w/w |
|---|---|---|
| Intragranular | | |
| Famotidine (micronized) | 40.00 | 13.6 |
| Fexofenadine | 60.00 | 20.3 |
| Melatonin | 4.00 | 1.4 |
| MCC | 34.50 | 11.7 |
| Heavy magnesium oxide | 25.00 | 8.5 |
| Calcium carbonate | 25.00 | 8.5 |
| L-Carnosine | 10.00 | 3.4 |
| Croscarmellose sodium | 14.00 | 4.7 |
| Simethicone powder | 25.00 | 8.5 |
| Binder solution | | |
| HPMC 5 cps | 15.00 | 5.1 |
| Water | QS | — |
| Extragranular | | |
| Pregelatinized starch | 30.00 | |
| Croscarmellose sodium | 10.00 | 3.4 |
| Colloidal Silicon Dioxide | 1.50 | 0.5 |
| Zinc stearate | 6.00 | 2.0 |
| Avg tablet weight | 300.00 | — |
| Coating | | |
| Opadry II 85F565369 | 9.00 | 3.0 |
| Water | q.s | — |
| Coated tablet weight | 309 | — |

Tablets were prepared using the process as detailed for batch ACGCP300102001A. The compressed tablets were then subjected to coating. Compression parameters are provided in Table 16 below. Characteristics of the coated tablets were then studied and same are provided in Table 17 below.

TABLE 16
Compression parameters

| Parameter | Observations |
|---|---|
| Tooling | 10.6 × 5.0 mm oval shape punches. |
| Average tablet weight (mg) | 300 mg |
| Tablet thickness (mm) | 5.7-5.9 |
| Tablet hardness (kP) | 4-6 |
| Disintegration time (min) | 5-6 |
| Remarks | No sticking of tablets during compression |

TABLE 17
Characteristics of coated tablets

| Container Closure | | |
|---|---|---|
| 60 cc HDPE bottle/33 mm CR closure with one 2 g silica gel bag | | |
| Test Parameters | | |
| Initial | 40° C./75% RH − 1M | 40° C./75% RH − 3M |

Assay (%)

| Famotidine | 97.1 | 97.2 | 98.4 |
|---|---|---|---|
| Fexofenadine | 97.3 | 99.9 | 98.0 |
| Melatonin | 97.6 | 98.3 | 98.5 |
| Water content | 4.88 | 3.56 | |
| Related Substances (%) | | | |
| Famotidine IMP-A | ND | ND | |
| Famotidine IMP-B | 0.04 | 0.03 | |
| Famotidine IMP-C | 0.01 | 0.02 | |
| Famotidine IMP-D | 0.05 | 0.03 | |
| Famotidine IMP-E | 0.13 | 0.08 | |
| Famotidine IMP-F | 0.00 | 0.01 | |
| Famotidine IMP-G | 0.05 | 0.06 | |
| Famotidine IMP-H | 0.10 | 0.03 | |
| Famotidine IMP-I | 0.01 | 0.06 | |
| Famotidine IMP-J | ND | ND | |
| Fexofenadine IMP-A | 0.05 | 0.04 | |
| Melatonin IMP-A | 0.01 | ND | |
| Any ukn highest of | 0.04 | 0.03 | |
| Famo @1.284 | | | |
| Any ukn highest of | 0.06 | 0.01 | |
| Fexo @ 1.241 | | | |
| Any ukn highest of | 0.14 | 0.03 | |
| Mela @0.714 | | | |
| Total Impurities | 0.96 | 0.64 | |
| Dissolution Methodology: Medium: 0.001N HCl, Speed: 50 RPM, | | | |
| Volume: 900 mL, Appts: USP II | | | |
| Time (min) | % drug release | % drug release | |
| Famotidine | | | |
| 5 | 38 | 38 | |
| 10 | 81 | 77 | |
| 20 | 98 | 93 | |
| 30 | 99 | 95 | |
| 45 | 100 | 97 | |
| 60 | 100 | 97 | |
| Fexofenadine | | | |
| 5 | 38 | 33 | |
| 10 | 78 | 70 | |
| 20 | 97 | 94 | |
| 30 | 99 | 96 | |
| 45 | 100 | 98 | |
| 60 | 100 | 99 | |
| Melatonin | | | |
| 5 | 36 | 38 | |
| 10 | 81 | 80 | |
| 20 | 99 | 96 | |
| 30 | 100 | 97 | |
| 45 | 100 | 98 | |
| 60 | 100 | 98 | |

Batch ACGCP300102008A

Coated tablets (using non-aqueous granulation) were prepared using the composition as provided in Table 18 below:

TABLE 18
Composition for Fexofenadine, Famotidine and Melatonin coated tablets

| Ingredients | mg/Unit | % w/w |
|---|---|---|
| Intragranular | | |
| Famotidine | 40.00 | 13.3 |
| Fexofenadine HCl | 60.00 | 20.0 |
| Melatonin | 4.00 | 1.3 |
| Microcrystalline cellulose | 34.50 | 16.0 |
| Heavy magnesium oxide | 25.00 | 8.3 |
| Calcium carbonate | 25.00 | 8.3 |
| L-Carnosine | 10.00 | 3.3 |
| Diglyceryl lauryl fumarate | 0.00 | — |
| Croscarmellose sodium | 14.00 | 4.7 |
| Binder solution | | |
| Hypromellose E5 | 15.00 | 5.0 |
| Simethicone | 25.00 | 8.3 |
| Isopropyl alcohol | q. s | — |
| Extra-granular | | |
| Croscarmellose sodium | 10.00 | 3.3 |
| Pregelatinized starch | 30.00 | |
| Colloidal Silicon Dioxide | 1.50 | 2.3 |
| Zinc stearate | 6.00 | 2.3 |
| Coating | | |
| Opadry II 85F565369 | 9.00 | 3.0 |
| Water | q. s | — |

Tablets were prepared using the process as detailed for batch ACGCP300102001A. The compressed tablets were then subjected to coating. Compression parameters are provided in Table 19 below. Characteristics of the coated tablets were then studied and same are provided in Table 20 below.

TABLE 19
Compression parameters

| Parameter | Observations |
|---|---|
| Tooling | 10.6 × 5.0 mm oval shape punches. |
| Average tablet weight (mg) | 300 mg |
| Tablet thickness (mm) | 5.1-5.2 |
| Tablet hardness (kP) | 4-6 |
| Disintegration time (min) | 9-12 |
| Remarks | No sticking of tablets during compression |

TABLE 20
Characteristics of coated tablets

| Container Closure | |
|---|---|
| 60 cc HDPE bottle /33 mm CR closure with one | |
| 2 g silica gel bag | |
| Test Parameters | |
| Initial | 40° C./75% RH − 3 M |

Assay (%)

| | | |
|---|---|---|
| Famotidine | 109.1 | 109.2 |
| Fexofenadine | 111.1 | 110.9 |
| Melatonin | 125.9 | 125.6 |
| Water content | 2.82 | |

Related Substances (%)

| | | |
|---|---|---|
| Famotidine IMP-A | ND | ND |
| Famotidine IMP-B | 0.06 | 0.06 |
| Famotidine IMP-C | 0.01 | 0.02 |
| Famotidine IMP-D | 0.03 | 0.04 |
| Famotidine IMP-E | 0.07 | 0.07 |
| Famotidine IMP-F | 0.01 | 0.00 |
| Famotidine IMP-G | 0.10 | 0.09 |
| Famotidine IMP-H | 0.10 | 0.03 |
| Famotidine IMP-I | 0.01 | 0.02 |
| Famotidine IMP-J | ND | ND |
| Fexofenadine IMP-A | 0.02 | 0.01 |
| Melatonin IMP-A | 0.01 | ND |
| Any ukn highest of Famo | 0.03 | 0.04 |
| @1.284 | | |
| Any ukn highest of Fexo @ | 0.07 | 0.01 |
| 1.241 | | |
| Any ukn highest of Mela | 0.05 | 0.04 |
| @0.714 | | |
| Total Impurities | 0.74 | 0.59 |

Dissolution Methodology: Medium: 0.001N HCl, Speed: 50 RPM, Volume: 900 mL,
Appts: USP II

| | | |
|---|---|---|
| Time (min) | % drug release | % drug release |

Famotidine

| | | |
|---|---|---|
| 5 | 28 | 36 |
| 10 | 63 | 72 |
| 20 | 98 | 100 |
| 30 | 105 | 103 |
| 45 | 107 | 105 |
| 60 | 109 | 105 |

Fexofenadine

| | | |
|---|---|---|
| 5 | 21 | 35 |
| 10 | 53 | 68 |
| 20 | 93 | 99 |
| 30 | 103 | 107 |
| 45 | 105 | 109 |
| 60 | 106 | 110 |

Melatonin

| | | |
|---|---|---|
| 5 | 39 | 63 |
| 10 | 88 | 109 |
| 20 | 116 | 122 |
| 30 | 118 | 124 |
| 45 | 119 | 124 |
| 60 | 119 | 124 |

Batch ACGCP300102009A

Tablets were prepared using the composition as provided in Table 21 below:

TABLE 21
Composition for Fexofenadine, Famotidine and Melatonin tablets

| Ingredients | mg/Unit | % w/w |
|---|---|---|
| Intragranular | | |
| Famotidine | 40.00 | 13.6 |
| Fexofenadine | 120.00 | 40.7 |
| Melatonin | 3.00 | 1.0 |
| MCC | 35.50 | 12.0 |
| Heavy magnesium oxide | 25.00 | 8.5 |
| Calcium carbonate | 25.00 | 8.5 |
| L-Carnosine | 10.00 | 3.4 |
| Croscarmellose sodium | 14.00 | 4.7 |
| Simethicone 30% emulsion | 25.00 | 8.5 |
| Binder solution | | |
| HPMC 5 cps | 15.00 | 5.1 |
| Water | QS | |
| Extragranular | | |
| Pregelatinized starch | 30.00 | 8.3 |
| Croscarmellose sodium | 10.00 | 3.4 |
| Colloidal Silicon Dioxide | 1.50 | 0.5 |
| Zinc stearate | 6.00 | 2.0 |
| Avg tablet weight | 360.00 | |

Tablets were prepared using the process as detailed for batch ACGCP300102001A. Compression parameters are provided in Table 22 below. Characteristics of the tablets were then studied and same are provided in Table 23 below.

TABLE 22
Compression parameters

| Parameter | Observations |
|---|---|
| Tooling | 10.6 × 5.0 mm oval shape punches. |
| Average tablet weight (mg) | 360 mg |
| Tablet hardness (kP) | 4-6 |
| Disintegration time (min) | 9-12 |
| Remarks | No sticking of tablets during compression |

TABLE 23
Characteristics of tablets

Test Parameters
Initial

Assay (%)

| | |
|---|---|
| Famotidine | 104.8 |
| Fexofenadine | 98.5 |
| Melatonin | 103.3 |
| Water content | |

Related Substances (%)

| Famotidine IMP-A | ND |
|---|---|
| Famotidine IMP-B | 0.00 |
| Famotidine IMP-C | 0.01 |
| Famotidine IMP-D | 0.01 |
| Famotidine IMP-E | 0.06 |
| Famotidine IMP-F | 0.01 |
| Famotidine IMP-G | 0.07 |
| Famotidine IMP-H | 0.02 |
| Famotidine IMP-I | 0.06 |
| Famotidine IMP-J | ND |
| Fexofenadine IMP-A | 0.04 |
| Melatonin IMP-A | 0.01 |
| Any ukn highest of Famo @1.284 | 0.05 |
| Any ukn highest of Fexo @ 1.241 | 0.11 |
| Any ukn highest of Mela @0.714 | 0.01 |
| Total Impurities | 0.69 |
| Dissolution Methodology: Medium: 0.001N HCl, Speed: 50 RPM, | |
| Volume: 900 mL, Appts: USP II | |
| Time (min) | % drug release |

Famotidine

| | |
|---|---|
| 5 | 31 |
| 10 | 73 |
| 20 | 94 |
| 30 | 98 |
| 45 | 101 |
| 60 | 102 |

Fexofenadine

| | |
|---|---|
| 5 | 20 |
| 10 | 53 |
| 20 | 82 |
| 30 | 96 |
| 45 | 100 |
| 60 | 101 |

Melatonin

| | |
|---|---|
| 5 | 34 |
| 10 | 81 |
| 20 | 96 |
| 30 | 98 |
| 45 | 99 |
| 60 | 99 |

Batch ACGCP300102010A

Tablets were prepared using the composition as provided in Table 24 below:

TABLE 24
Composition for Fexofenadine, Famotidine and Melatonin tablets

| Ingredients | mg/Unit | % w/w |
|---|---|---|
| Intragranular | | |
| Famotidine | 40.00 | 13.6 |
| Fexofenadine | 120.00 | 40.7 |
| Melatonin | 3.00 | 1.0 |
| MCC | 35.50 | 12.0 |
| Heavy magnesium oxide | 25.00 | 8.5 |
| Calcium carbonate | 25.00 | 8.5 |
| L-Carnosine | 10.00 | 3.4 |
| Croscarmellose sodium | 14.00 | 4.7 |
| Simethicone 30% emulsion | 25.00 | 8.5 |
| Binder solution | | |
| HPMC 5 cps | 15.00 | 5.1 |
| Water | QS | — |
| Extragranular | | |
| Pregelatinized starch | 30.00 | 8.3 |
| Croscarmellose sodium | 10.00 | 3.4 |
| Colloidal Silicon Dioxide | 1.50 | 0.5 |
| Zinc stearate | 6.00 | 2.0 |
| Avg tablet weight | 360.00 | — |

Tablets were prepared using the process as detailed for batch ACGCP300102001A. Compression parameters are provided in Table 25 below. Characteristics of the tablets were then studied and same are provided in Table 26 below.

TABLE 25
Compression parameters

| Parameter | Observations |
|---|---|
| Tooling | 10.6 × 5.0 mm oval shape punches. |
| Average tablet weight (mg) | 360 mg |
| Tablet hardness (kP) | 4-6 |
| Disintegration time (min) | 9-12 |
| Remarks | No sticking of tablets during compression |

TABLE 26
Characteristics of tablets

Test Parameters
Initial

Assay (%)

| Famotidine | 101.5 |
|---|---|
| Fexofenadine | 98.6 |
| Melatonin | 99.2 |
| Water content | 4.78 |
| Dissolution Methodology: Medium: 0.001N HCl, Speed: 50 RPM, | |
| Volume: 900 mL, Appts: USP II | |
| Time (min) | % drug release |

Famotidine

| | |
|---|---|
| 5 | 60 |
| 10 | 83 |
| 20 | 95 |
| 30 | 97 |
| 45 | 99 |
| 60 | 100 |

Fexofenadine

| | |
|---|---|
| 5 | 48 |
| 10 | 69 |
| 20 | 85 |
| 30 | 92 |
| 45 | 97 |
| 60 | 99 |

Melatonin

| | |
|---|---|
| 5 | 74 |
| 10 | 96 |
| 20 | 100 |
| 30 | 100 |
| 45 | 101 |
| 60 | 101 |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

1. A pharmaceutical composition comprising: Fexofenadine or salt or hydrates or solvates thereof, Famotidine or salt or hydrates or solvates thereof and Melatonin or salt or hydrates or solvates thereof.

2. The composition of claim 1, wherein the composition comprises Fexofenadine or salt or hydrates or solvates thereof, Famotidine or salt or hydrates or solvates thereof and Melatonin or salt or hydrates or solvates thereof in a weight ratio ranging from 1:1:1 to 100:50:1.

3. The composition of claim 1, wherein the composition comprises Fexofenadine or salt or hydrates or solvates thereof, Famotidine or salt or hydrates or solvates thereof and Melatonin or salt or hydrates or solvates thereof in a weight ratio ranging from 4:2:1 to 50:25:1.

4. The composition of claim 1, wherein Fexofenadine or salt or hydrates or solvates thereof is present in an amount ranging from 20 mg to 500 mg.

5. The composition of claim 1, wherein Famotidine or salt or hydrates or solvates thereof is present in an amount ranging from 10 mg to 100 mg.

6. The composition of claim 1, wherein Melatonin or salt or hydrates or solvates thereof is present in an amount ranging from 1 mg to 80 mg.

7. The composition of claim 1, wherein the composition further comprises a pharmaceutically acceptable excipient.

8. The composition of claim 7, wherein the pharmaceutically acceptable excipient is selected from any or a combination of: a diluent, an anti-oxidant, a preservative, an alkalizing agent, a buffering agent, a disintegrant, a binder, an anti-foaming agent, a solvent, a glidant, a lubricant, a flavoring agent, a sweetener, a coating agent, a rate controlling polymer or non-polymer, a zinc salt, a fatty acid or derivative thereof, an amino acid or metabolites or amino acid derivatives, a bulking agent, an anti-tacking agent, an emulsifier, a surfactant, a plasticizer and a stabilizer.

9. The composition of claim 8, wherein the fatty acid or derivative thereof comprises any or a combination of: diglyceryl lauryl fumarate, diglyceryl lauryl succinate, and diglyceryl capryl succinate.

10. The composition of claim 1, wherein the composition comprises an intra-granular portion and an extra-granular portion, wherein the intra-granular portion comprises Fexofenadine or salt or hydrates or solvates thereof, Famotidine or salt or hydrates or solvates thereof, Melatonin or salt or hydrates or solvates thereof and a pharmaceutically acceptable excipient, and the extra-granular portion comprises a pharmaceutically acceptable excipient.

11. The composition of claim 1, wherein the composition comprises an intra-granular portion and an extra-granular portion, wherein the intra-granular portion comprises Famotidine or salt or hydrates or solvates thereof, Melatonin or salt or hydrates or solvates thereof and a pharmaceutically acceptable excipient, and the extra-granular portion comprises Fexofenadine or salt or hydrates or solvates thereof and a pharmaceutically acceptable excipient.

12. The composition of claim 1, wherein the composition comprises an intra-granular portion and an extra-granular portion, wherein the intra-granular portion comprises Fexofenadine or salt or hydrates or solvates thereof, Melatonin or salt or hydrates or solvates thereof and a pharmaceutically acceptable excipient, and the extra-granular portion comprises Famotidine or salt or hydrates or solvates thereof and a pharmaceutically acceptable excipient.

13. The composition of claim 1, wherein the composition comprises an intra-granular portion and an extra-granular portion, wherein the intra-granular portion comprises Fexofenadine or salt or hydrates or solvates thereof and Famotidine or salt or hydrates or solvates thereof and a pharmaceutically acceptable excipient, and the extra-granular portion comprises Melatonin or salt or hydrates or solvates thereof and a pharmaceutically acceptable excipient.

14. The composition of claim 1, wherein the composition comprises an intra-granular portion and an extra-granular portion, and wherein the portions are compressed together to obtain a tablet dosage form, optionally coated with a seal coat.

15. The composition of claim 1, wherein the composition comprises an intra-granular portion and an extra-granular portion, and wherein the intra-granular portion comprises Fexofenadine hydrochloride in an amount of 60 mg, Famotidine in an amount of 40 mg, Melatonin in an amount of 4 mg, and a pharmaceutically acceptable excipient, and the extra-granular portion comprises a pharmaceutically acceptable excipient.

16. The composition of claim 1, wherein the composition comprises an intra-granular portion and an extra-granular portion, and wherein the intra-granular portion comprises Fexofenadine hydrochloride in an amount of 120 mg, Famotidine in an amount of 40 mg, Melatonin in an amount of 3 mg, and a pharmaceutically acceptable excipient, and the extra-granular portion comprises a pharmaceutically acceptable excipient.

17. A method of treating oral and gastrointestinal disorders in a patient in need thereof comprising administering to the patient a therapeutically effective amount of a pharmaceutical composition of claim 1.

18. Use of a pharmaceutical composition of claim 1 in preparation of a medicament for treatment of any of a dermatological disease, an inflammation associated with COVID 19, a gastrointestinal disease and a sleep disorder in a patient in need thereof.
